SEQUENCE LISTING (9 sequences)

SEQ ID NO: 1 - DNA, 3743 bp, Homo sapiens
  CDS (481)..(1728)
ttgcgggaaagagccaaaccctggcgttggggggcccgggcggggagcccctcccgcggtccacagcgacgcctgcccagccctcctccccttccggctccggcacggggccccgaggcgttcggaggccaggcgggtttctgtcaggcccggggaggaggggcgggcggggcggccgctgcctccccgggacgggccgtaccacgcggacggggaggacggggccaggggactgcagggcggctgcaccgcccgggggcggggtgcggacgggccggcgggctccccggggcggggcgggagggcggggcgtggggcggacggaaccaccggggcggggtgggaggtaacgggacgggcgcgaccatggcgcggtgagggagcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgagttcttggcacagctgaagatcatggactacagcctgctggtgggcatccacgacgtggaccgggcagagcaggaggagatggaggtggaggagcgggcagaggacgaggagtgtgagaatgatggggtgggtggcaacctactctgctcctatggcacacctccggacagccctggcaacctcctcagctttcctcggttctttggtcctggggaattcgacccctctgttgacgtctatgccatgaaaagccatgaaagttcccccaagaaggaggtgtatttcatggccatcattgatatcctcacgccatacgatacaaagaagaaagctgcacatgctgccaaaacggtgaaacacggggcaggggccgagatctcgactgtgaaccctgagcagtactccaaacgcttcaacgagtttatgtccaacatcctgacgtagttctcttctaccttcagccgagaccgagagactggatatggggtcggggatcgggacttagggagaagggtgtatttgggctagatgggagggtgggagcgagatcgggtttgggagggctttagcaatgagacttgcagcctgtgacaccgaaagagactttagctgaagaggagggggatgtgctgtgtgtgcaccagctcacaggatgtaaccccaccttctgcttacccttgattttttctccccatttgacacccaggttaaaaaggggttccctttttggtaccttgtaaccttttaagataccttggggctagagatgacttcgtgggtttatttgggttttgtttctgaaatttcattgctccaggtttgctatttataatcatatttcatcagcctacccaccctccccatctttgctgctctcagttcccttcaattaaagagatacccagtagacccagcacaagggtccttccagaaccaagtgctatggatgccagattggagaggtcagacacctcgccctgctgcatttgctcttgtctggattaactttgtaatttatggagtattgtgcacaacttcctccacctttcccttggattcaagtgaaaactgttgcattattcctccatcctgtctggaatacaccaggtcaacaccagagatctcagatcagaatcagagatctcagaggggaataagttcatcctcatgggatggtgaggggcaggaaagcggctgggctcttggacaccctggttctcagagaaccctgtgatgatcacccaagccccaggctgtcttagcccctggagttcagaagtcctctctgtaaatcctgcctcccactaggtcaagaggaactagagtacctttggatttatcaggaccctcatgtttaaatggttatttccctttgggaaaacttcagaaactgatgtatcaaatgaggccctgtgccctcgatctatttccttcttccttctgacctcctcccaggcactcttacttctagccgaactcttagctctgggcagatctccaagcgcctggagtgctttttagcagagacacctcgttaagctccgggatgaccttgtaggagatctgtctcccctgtgcctggagagttacagccagaaaggtgcccccatcttagagtgtggtgtccaaacgtgaggtggcttcctagttacatgaggatgtgatccaggaaatccagtttggaggcttgatgtgggttttgacctggcctcaccttggggctgtttttccttgttgccccgctctagacttttagcagatctgcacccacaggcttttttggaaggagtggcttcctcgaggtgttccacctgcttcggagcctgccacccaggccctcagaactgaccacaggctgctctggccaggagagaaacagctctgttgttctgcattgggggaggtacattcctgcatcttctcaccccctcaaccaggaactggggatttgggatgagatatggtcagacttgtagataaccccaaagatgtgaagatcgcttgtgaaaccattttgaatgaatagattggtttcctgtggctccctccaaacctggccaagcccagcttccgaagcaggaaccagcactgtctctgtgcctgactcacagcatataggtcaggaaagaatggagacggcattcttggacttcactggggctgctggattggatgggaaaccttctggaagaggcagatgggggtcaaaccactgccttgccccaggaaggggccataggtaggtctgaacaactgccggaagaccactacatgacttagggaacttgaaaccaactggctcatggagaaaacaaatttgacttgggaaagggattatgtaggaataatgtttggacttgatttccccacgtcataatgaagaatggaagtttggatctgctcctcgtcaggcgcagcatctctgaagcttggaaagctgtcttccagggttgtaa

SEQ ID NO: 2 - protein, 416 aa, Homo sapiens
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT

SEQ ID NO: 3 - DNA, 23 bp, Mus musculus
accatcccaaagcacccaggacc

SEQ ID NO: 4 - DNA, 24 bp, Mus musculus
cgtgcgtatgccgtcgtcgtttcc

SEQ ID NO: 5 - DNA, 24 bp, Mus musculus
agaagcgagaagcgaactgattgg

SEQ ID NO: 6 - DNA, 29 bp, Mus musculus
cctcgtcctctgcccgctcctccacctcc

SEQ ID NO: 7 - DNA, 27 bp, Mus musculus
cgccagcaagacaagaccaagaagaag

SEQ ID NO: 8 - DNA, 24 bp, Mus musculus
cgctcctccacctccatctcctcc

SEQ ID NO: 9 - DNA, 21 bp, Mus musculus
gcatccttcagccccttgttg

(19) United States
(12) Patent Application Publication
(10) Pub. No.: US 2006/0089320 A1
(43) Pub. Date: Apr. 27, 2006

(54) Modulation of type IIß phosphoinositide phosphate kinase

(76) Inventors: Lewis C Cantley, Cambridge, MD (US); Katja A Lamia, Brookline, MA (US); Lucia F.E.R Plant Rameh, Belmont, MA (US); Barbara Kahn, Cambridge, MD (US); Odile Peroni, Brookline, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/502,993

(22) PCT Filed: Feb. 3, 2003
(86) PCT No.: PCT/US03/03065

§ 371 Date: Aug. 24, 2005

Related U.S. Application Data
(60) Provisional application No. 60/353,758, filed on Feb. 1, 2002.

(51) Int. Cl. A61K 48/00; A61K 31/426; A61K 31/155; A61K 31/175

(52) U.S. Cl. 514/44; 514/369; 514/592; 514/635

(57) ABSTRACT

The invention provides methods for modulating type IIβ phosphoinisitide phosphate kinase (PIPKIIβ) activity for treating PIPKIIβ-associated disorders. The invention also provides methods of identifying candidate agents for treating PIPKIIβ-associated disorders.

### RELATED APPLICATIONS

This application claims the benefit under 35 U.S.C. § 119(e) of U.S. provisional application 60/353,758, filed Feb. 1, 2002, the entire disclosure of which is incorporated herein by reference.

### GOVERNMENT SUPPORT

This invention was made in part with government support under grant number RO1 GM36624 from the National Institutes of Health (NIH) and under grant number 5P30-DK36836-14 from the National Institute of Diabetes and Digestive and Kidney Diseases (NIDDK) of the National Institutes of Health. The government may have certain rights in this invention.

### FIELD OF THE INVENTION

The invention relates to modulation of type II, phosphoinositide phosphate kinase (PIPKIIβ) activity for treating PIPKIIβ-associated disorders. In addition, the invention relates to the use of PIPKIIβ nucleic acid molecules and polypeptides for diagnosis, monitoring and treatment of PIPKIIβ-associated disorders. The invention also relates to screening for agents that modulate PIPKIIβ activity, which are useful in the treatment of PIPKIIβ-associated disorders.

### BACKGROUND OF THE INVENTION

Until recently, the type II phosphoinositide phosphate kinases were thought to produce phosphatidylinositol-4,5-bisphosphate (PI4,5P₂) by phosphorylating the 5 position of phosphatidylinositol-4-phosphate (PI4P). However, in 1997 these enzymes were shown to be in a novel, previously unknown, pathway that involves production of PI4,5P₂ from phosphatidylinositol-5-phosphate (PI5P) (Rameh et al., 1997). The enzymes for this pathway are conserved from mammals to worms, but the importance for biological function is not known.

It is now known that the type II PIP kinases produce phosphatidyl inositol 4,5 bisphosphate (PI4,5P₂) by phosphorylating the 4^{th} position of the inositol ring of phosphatidyl inositol 5-phosphate (PI5P) (Rameh et. al., 1997). The majority of PI4,5P₂ is produced by the type I PIP kinases (PIPKIβ), which phosphorylate the 5^{th} position of the inositol ring of phosphatidyl inositol 4-P (PI4P). The evolution of these two pathways for synthesis of PI4,5P₂ appears to be quite ancient in that both type I and type II PIP kinases are found not only in vertebrates but also in worms and flies. Mammals have three isoforms of type II PIP kinase encoded by distinct genes: PIPKIIα (Divecha et al., 1995), PIPKIIβ (Castellino et al., 1997), and PIPKIIγ (Itoh et al., 1998). These enzymes have different, but somewhat overlapping tissue distributions.

The reason that two pathways evolved for production of PI4,5P₂ is not known. It is possible that type II PIP kinases generate PI4,5P₂ at a unique location in the cell for a specific purpose. PI4,5P₂ is known to play many roles in the cell: it is a precursor for several second messengers (Toker 1998) and can interact with a variety of proteins that affect the actin cytoskeleton (for review, see Takenawa and Miki, 2001). Experiments with fluorescently tagged PH domains that specifically target PI4,5P₂ have suggested that local populations of “free” PI4,5P₂ are regulated by various signaling events (for review see Martin, 2001). PI4,5P₂ has also been shown to effect the localization of the tubby protein which was originally discovered as the gene responsible for an obesity phenotype in a strain of mice with a spontaneous mutation in the tubby locus (Santagata et. al., 2001). Tubby is localized to the plasma membrane via its SH2 domain which binds PI4,5P₂. It is released from the membrane upon activation of phospholipase C beta (PLCβ) which cleaves PI4,5P₂ to form diacylglycerol (DAG) and inositol 1,4,5-trisphosphate (IP₃) in response to activation of G protein coupled receptors. This is another example of a discrete pool of PI4,5P₂ which may be regulated by the alternative pathway.

Alternatively, the importance of type II PIP kinases may be to reduce the level of the less understood lipid, PI5P. A signaling role for PI5P has not yet been described. However, a variety of protein domains have evolved the ability to bind to specific phosphoinositides as a mechanism of localization at specific membranes (for review see Wishart et. al 2001; Hurley and Meyer 2001; Lemmon and Ferguson 2000, Gillooly et. al, 2001) and it is possible that PI5P mediates the recruitment of specific proteins to the membrane.

Phosphoinositides also play a crucial role in insulin signaling. The insulin receptor activates phosphoinositide 3-kinase (PI3K) to produce the second messenger phosphatidylinositol-3,4,5-trisphosphate (PIP₃). This lipid recruits a set of proteins to the membrane, including the protein-Ser/Thr kinase Akt (also known as protein kinase B). Activation of PI3K and Akt are required for most insulin responses that have been investigated, including inhibition of glycogen synthase kinase 3 (GSK3) and the activation of glucose transport.

PIP₃ levels are regulated not only by their rate of production by PI3K but also by their rate of destruction by phosphatases. The SH2-domain-containing inositol phosphatases SHIP1 and SHIP2 degrade PIP₃ by dephosphorylating the 5^{th} position of the inositol ring to produce phosphatidylinositol 3,4-bisphosphate (PI3,4P₂). SHIP2 knockout mice are severely hypersensitive to insulin, as one would expect if they have increased PIP₃ levels produced at sites of insulin receptor activation (Clement et al., 2001).

Insulin signaling and appropriate insulin response in patients has been identified as a factor in diseases such as type II diabetes and obesity. Insulin insensitivity or reduced insulin sensitivity in a patient may result in adult-onset diabetes (type II diabetes) and/or can contribute to obesity, both of which may have severe clinical consequences for the individual. An estimated 15.7 million Americans have diabetes, and individuals with adult-onset, type 2, diabetes represent 90 to 95 percent of all diabetics. Almost one-third of all diabetics in the U.S. are unaware that they have the disorder, and undetected and uncontrolled diabetes can have serious side effects, such as blindness, heart disease, nerve disease, and kidney disease.

Obesity also has numerous risks for patients and may result in premature mortality. Obesity affects at least 39 million Americans: more than one-quarter of all adults and about one in five children. Each year, obesity causes at least 300,000 excess deaths in the U.S. and costs the country more than $100 billion. Obesity is the second leading cause of unnecessary deaths in the U.S.

In addition to the increased clinical risks, both obesity and type II diabetes may also result in a reduced quality of life for the affected individual. Because type II diabetes and obesity are major disorders in current society, which have serious health and life quality consequences, improved methods of treatment and/or reliable diagnosis are needed and would be beneficial for patients and their families and health-care providers.

### SUMMARY OF THE INVENTION

The invention relates in part to methods of increasing insulin sensitivity in patients and provides methods for treating disorders such as type II diabetes, obesity, excess fat accumulation and reduced sensitivity to insulin.

According to one aspect of the invention, methods of treating a subject having or suspected of having type II diabetes are provided. The methods include administering to a subject in need of such treatment an effective amount of an agent that reduces the activity of PIPKIIβ in the subject, as a treatment for the type II diabetes. In some embodiments the method further includes administering a pharmaceutical agent that increases sensitivity of tissues to insulin to the subject. In certain embodiments, the pharmaceutical agent is selected from the group consisting of: metformin, pioglitazone, and rosiglitazone. In some embodiments, the method further includes administering a pharmaceutical agent that increases insulin release. In some embodiments, the pharmaceutical agent is selected from the group consisting of sulfonylureas, nateglinide and repaglinide. In some embodiments, the sulfonylurea is selected from the group consisting of: glibenclamide (glyburide), gliclazide and glimepiride. In some embodiments, the method further includes administering insulin to the subject. In some embodiments, agent is a PIPKIIβ inhibitor. In certain embodiments, the agent is an PIPKIIβ antisense sequence.

According to another aspect of the invention, methods of treating a subject having or suspected of having reduced insulin sensitivity are provided. The methods include administering to a subject in need of such treatment an effective amount of an agent that reduces the activity of PIPKIIβ in the subject, as a treatment for the reduced insulin sensitivity. In some embodiments, the method also includes administering a pharmaceutical agent that increases sensitivity of tissues to insulin to the subject. In certain embodiments, the pharmaceutical agent is selected from the group consisting of: metformin, pioglitazone, and rosiglitazone. In some embodiments, the method further includes administering a pharmaceutical agent that increases insulin release. In some embodiments, the pharmaceutical agent is selected from the group consisting of sulfonylureas, nateglinide and repaglinide. In some embodiments, the sulfonylurea is selected from the group consisting of: glibenclamide (glyburide), gliclazide and glimepiride. In some embodiments, the method further includes administering insulin to the subject. In some embodiments, the agent is a PIPKIIβ inhibitor. In some embodiments, the agent is a PIPKIIβ antisense sequence.

According to another aspect of the invention, methods of treating a subject having or suspected of having obesity are provided. The methods include administering to a subject in need of such treatment an effective amount of an agent that reduces the activity of PIPKIIβ in the subject, as a treatment for the obesity. In some embodiments, methods also include administering a pharmaceutical agent that increases sensitivity of tissues to insulin to the subject. In certain embodiments, the agent is a PIPKIIβ inhibitor. In other embodiments, the agent is an PIPKIIβ antisense sequence.

According to yet another aspect of the invention, methods of treating a subject having or suspected of having excess fat accumulation are provided. The methods include administering to a subject in need of such treatment an effective amount of an agent that reduces the activity of PIPKIIβ in the subject, as a treatment for the excess fat accumulation. In some embodiments, methods also include administering a pharmaceutical agent that increases sensitivity of tissues to insulin to the subject. In certain embodiments, the agent is a PIPKIIβ inhibitor. In other embodiments, the agent is an PIPKIIβ antisense sequence.

According to another aspect of the invention methods of treating a subject having or suspected of having an increased sensitivity to insulin are provided. The methods include administering to a subject in need of such treatment an agent that increases the activity of PIPKIIβ in the subject, as a treatment for increased sensitivity to insulin.

According to yet another aspect of the invention, methods for identifying an agent that decreases PIPKIIβ activity are provided. The methods include determining a first amount of activity of a PIPKIIβ polypeptide, contacting the PIPKIIβ polypeptide with a candidate pharmacological agent, determining the amount of activity of the contacted PIPKIIβ polypeptide, wherein a decrease in the amount of activity of the contacted PIPKIIβ polypeptide relative to the first amount of activity of the PIPKIIβ polypeptide is an indication that the candidate pharmacological agent decreases PIPKIIβ activity.

According to another aspect of the invention, methods for identifying an agent that increases PIPKIIβ activity, are provided. The methods include determining a first amount of activity of a PIPKIIβ polypeptide, contacting the PIPKIIβ polypeptide with a candidate pharmacological agent, determining the amount activity of the contacted PIPKIIβ polypeptide, wherein an increase in the amount of activity in the contacted PIPKIIβ polypeptide relative to the first amount of activity of the PIPKIIβ polypeptide is an indication that the candidate pharmacological agent increases PIPKIIβ activity.

According to another aspect of the invention, methods of diagnosing a PIPKIIβ-associated disorder in a subject are provided. The methods include obtaining a biological sample from a subject, determining the level of activity of a PIPKIIβ polypeptide molecule in the biological sample, comparing the level of activity of the PIPKIIβ polypeptide molecule in the biological sample with the level of activity of a PIPKIIβ polypeptide molecule in a control tissue, wherein a higher level of activity of the PIPKIIβ polypeptide molecule in the biological sample from the subject than the activity of the PIPKIIβ polypeptide molecule in the control sample is diagnostic for a PIPKIIβ-associated disorder in the subject. In some embodiments, the biological sample is selected from the group consisting of: tissue and cells. In some embodiments, the tissue or cells is selected from the group consisting of: skeletal muscle, brain, and adipose tissue. In certain embodiments, the activity is determined with a kinase assay. In some embodiments, the PIPKIIβ-associated disorder is selected from the group consisting of: diabetes and obesity.

In some embodiments of the foregoing aspects of the invention the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1. In some embodiments of the foregoing aspects of the invention, the PIPKIIβ polypeptide comprises an amino acid sequence set forth as SEQ ID NO: 2.

According to another aspect of the invention, methods for preparing an animal model of a disorder characterized by increased activity of a PIPKIIβ molecule are provided. The methods include introducing into a non-human subject a PIPKIIβ molecule that increases PIPKIIβ activity. In some embodiments, the PIPKIIβ molecule is a PIPKIIβ nucleic acid molecule. In some embodiments, the PIPKIIβ nucleic acid molecule comprises a nucleotide sequence set forth as SEQ ID NO: 1, or having at least about 95% homology to the nucleotide sequence set forth as SEQ ID NO:1. In certain embodiments, the PIPKIIβ molecule is a PIPKIIβ polypeptide. In some embodiments, the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1. In some embodiments, the PIPKIIβ polypeptide comprises an amino acid sequence set forth as SEQ ID NO: 2. In some embodiments, the animal model is of a disorder that is selected from the group consisting of: type II diabetes, insensitivity to insulin, excess fat accumulation, and obesity.

According to another aspect of the invention, methods for preparing an animal model of a disorder characterized by decreased expression of a PIPKIIβ molecule are provided. The methods include introducing into a non-human subject, a mutant PIPKIIβ molecule, that decreases PIPKIIβ activity. In some embodiments, the PIPKIIβ molecule is a mutant PIPKIIβ nucleic acid molecule. In certain embodiments, the PIPKIIβ molecule is a mutant PIPKIIβ polypeptide.

According to another aspect of the invention, methods for evaluating the effect of a candidate pharmacological agent on a PIPKIIβ-associated disorder are provided. The methods include administering a candidate pharmaceutical agent to a subject with a PIPKIIβ-associated disorder; determining the effect of the candidate pharmaceutical agent on the activity level of a PIPKIIβ polypeptide relative to the activity level of a PIPKIIβ polypeptide in a subject to which no candidate pharmaceutical agent is administered, wherein a relative increase or relative decrease in the activity level of the PIPKIIβ polypeptide indicates an effect of the pharmaceutical agent on the PIPKIIβ-associated disorder. In some embodiments, the activity level of the PIPKIIβ polypeptide is determined with a kinase assay. In some embodiments, the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1. In certain embodiments, the PIPKIIβ polypeptide comprises an amino acid sequence set forth as SEQ ID NO: 2. In some embodiments, the PIPKIIβ-associated disorder is selected from the group consisting of: type II diabetes, insensitivity to insulin, excess fat accumulation, and obesity.

According to yet another aspect of the invention, methods for evaluating the effect of a candidate pharmacological agent on a PIPKIIβ-associated disorder are provided. The methods include administering a candidate pharmaceutical agent to a subject with a PIPKIIβ-associated disorder; determining the effect of the candidate pharmaceutical agent on the level of expression of a PIPKIIβ molecule relative to the level of expression of a PIPKIIβ molecule in a subject to which no candidate pharmaceutical agent is administered, wherein a relative increase or relative decrease in the level of expression of a PIPKIIβ molecule indicates an effect of the pharmaceutical agent on the PIPKIIβ-associated disorder. In some embodiments, the PIPKIIβ molecule is a PIPKIIβ nucleic acid molecule. In certain embodiments, the PIPKIIβ nucleic acid molecule comprises a nucleotide sequence set forth as SEQ ID NO: 1, or having at least about 95% homology to the nucleotide sequence set forth as SEQ ID NO:1. In some embodiments, the PIPKIIβ molecule is a PIPKIIβ polypeptide. In some embodiments, the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1. In certain embodiments, the PIPKIIβ polypeptide comprises an amino acid sequence set forth as SEQ ID NO: 2. In some embodiments, the animal model is of a disorder that is selected from the group consisting of: type II diabetes, insensitivity to insulin, excess fat accumulation, and obesity.

According to another aspect of the invention, methods of diagnosing a PIPKIIβ-associated disorder in a subject are provided. The methods include obtaining a biological sample from a subject, determining the level of expression of a PIPKIIβ nucleic acid molecule in the biological sample, comparing the level of expression in the biological sample with the level of expression of the nucleic acid molecule in a control biological sample, wherein a higher level of expression of the PIPKIIβ nucleic acid molecule in the biological sample from the subject than in the control biological sample is diagnostic for a PIPKIIβ-associated disorder in the subject.

According to another aspect of the invention, methods for determining progression or regression of a PIPKIIβ-associated disorder in a subject are provided. The methods include obtaining from a subject two biological samples, wherein the samples comprise the same tissue type and are obtained at different times, determining a level of expression of a PIPKIIβ nucleic acid molecule in the two biological samples, and comparing the levels of expression in the two biological samples, wherein a higher level of expression of the PIPKIIβ nucleic acid molecule in the first biological sample than in the second biological sample indicates regression of a PIPKIIβ-associated disorder, wherein a lower level of expression of the PIPKIIβ nucleic acid molecule in the first biological sample than the second biological sample indicates progression of a PIPKIIβ-associated disorder.

In some embodiments of the foregoing aspects of the invention, the PIPKIIβ nucleic acid molecule comprises a nucleotide sequence set forth as SEQ ID NO: 1, or having at least about 95% homology to the nucleotide sequence set forth as SEQ ID NO:1. In some embodiments of the foregoing aspects of the invention the biological sample is selected from the group consisting of: tissue and cells. In certain embodiments of the foregoing aspects of the invention the tissue or cells is selected from the group consisting of: skeletal muscle, brain, and adipose tissue. In some embodiments of the foregoing aspects of the invention the PIPKIIβ-associated disorder is selected from the group consisting of: type II diabetes, insensitivity to insulin, excess fat accumulation, and obesity. In some embodiments of the foregoing aspects of the invention the level of expression of PIPKIIβ nucleic acid molecules is determined by a method selected from the group consisting of nucleic acid hybridization and nucleic acid amplification. In certain embodiments of the foregoing aspects of the invention the nucleic acid hybridization is performed using a nucleic acid microarray. In some embodiments of the foregoing aspects of the invention the nucleic acid amplification is selected from the group consisting of PCR, RT-PCR, and real-time PCR.

According to another aspect of the invention, methods of diagnosing a PIPKIIβ-associated disorder in a subject are provided. The methods include obtaining a biological sample from a subject, comparing the level of PIPKIIβ polypeptide in the biological sample with the level of PIPKIIβ polypeptide in a control biological sample, wherein a level of PIPKIIβ polypeptide in the biological sample from the subject that is higher than the level of PIPKIIβ polypeptide in the control biological sample is diagnostic for a PIPKIIβ-associated disorder in the subject.

According to yet another aspect of the invention, methods for determining progression or regression of a PIPKIIβ-associated disorder in a subject are provided. The methods include obtaining from a subject two biological samples, wherein the samples comprise the same tissue type and are obtained at different times, comparing the levels of PIPKIIβ polypeptide in the two biological samples, wherein a higher level of PIPKIIβ polypeptide in the first biological sample than in the second biological sample indicates regression of a PIPKIIβ-associated disorder, wherein a lower level of PIPKIIβ polypeptide in the first biological sample than the second biological sample indicates progression of a PIPKIIβ-associated disorder.

In some embodiments of the foregoing aspects of the invention, the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1. In certain embodiments of the foregoing aspects of the invention, the PIPKIIβ polypeptide comprises an amino acid sequence set forth as SEQ ID NO: 2. In some embodiments of the foregoing aspects of the invention, wherein the biological sample is selected from the group consisting of: tissue and cells. In some embodiments of the foregoing aspects of the invention, the tissue or cells is selected from the group consisting of: skeletal muscle, brain, and adipose tissue. In some embodiments of the foregoing aspects of the invention, the PIPKIIβ-associated disorder is selected from the group consisting of: type II diabetes, insensitivity to insulin, excess fat accumulation, and obesity. In certain embodiments of the foregoing aspects of the invention, the level of expression of the PIPKIIβ polypeptide is determined by a method selected from the group consisting of immunohistochemistry and immunoprecipitation.

According to yet another aspect of the invention, methods of diagnosing a PIPKIIβ-associated disorder in a subject are provided. The methods include obtaining a biological sample from a subject, determining the nucleotide sequence of a PIPKIIβ nucleic acid molecule in the biological sample, comparing the nucleotide sequence in the subject sample with the nucleotide sequence of a control PIPKIIβnucleic acid molecule, wherein a difference between the nucleotide sequence in the subject biological sample and the control PIPKIIβ nucleic acid molecule is diagnostic for a PIPKIIβ-associated disorder in the subject. In some embodiments, the PIPKIIβ nucleic acid molecule comprises a nucleotide sequence set forth as SEQ ID NO: 1, or having at least about 95% homology to the nucleotide sequence set forth as SEQ ID NO:1. In certain embodiments, the biological sample is selected from the group consisting of: tissue and cells. In some embodiments, the tissue or cells is selected from the group consisting of: skeletal muscle, brain, and adipose tissue. In some embodiments, the PIPKIIβ-associated disorder is selected from the group consisting of: type II diabetes, insensitivity to insulin, excess fat accumulation, and obesity.

In another aspect, the invention provides for use of the foregoing agents, compounds and molecules in the preparation of medicaments also is provided, particularly medicaments for the treatment of diabetes, obesity and reduced insulin sensitivity.

These and other aspects of the invention are described further below.

### BRIEF DESCRIPTION OF THE FIGURES

FIG. 1 is a bar graph comparing the percent fat tissue in PIPKIIβ^{-/-} male mice and wild-type male mice at 26 weeks of age.

FIG. 2 is a bar graph comparing the percent of fat tissue in PIPKIIβ^{-/-} male mice and wild-type male mice at 10 weeks of age when fed a high fat diet.

FIG. 3 is a bar graph comparing the percent of fat tissue in PIPKIIβ^{-/-} male mice and wild-type male mice at 36 weeks of age.

FIG. 4 is a set of graphs depicting results of insulin tolerance tests of male mice at 8 weeks, 16, weeks, and 24 weeks of age showing that PIPKIIβ^{-/-} do not develop age-onset insulin resistance while there wild-type counterparts do develop insulin resistance.

FIG. 5 contains graphs showing that female knockout mice (FIG. 5A) have a similar amount of body fat as their wild-type counterparts but are significantly more sensitive to insulin than their wild type littermates at 24 weeks of age (FIG. 5B).

FIG. 6 is a digitized image of a immunoblot showing that PIP4K type II overexpression negatively regulates insulin signaling. Ha-Akt was immunoprecipitated from lysates and blotted with anti-pT308 antibody to determine its activation state.

FIG. 7 is a bar graph depicting the reduction in the levels of cellular PtdIns-3,4,5-P₃ resulting from overexpression of PIP4k type II and Ship2.

### DETAILED DESCRIPTION OF THE INVENTION

To determine a physiological role for the pathway that involves production of PI4,5P₂ from phosphatidylinositol-5-phosphate (PIP5), mice were generated that had impaired expression of PIPKIIβ, a type II PIPK enzyme highly expressed in muscle. Surprisingly, the PIPKIIβ^{-/-} mice were hypersensitive to insulin when compared to wild type littermates. Male knockout mice unexpectedly also accumulated less body fat than wild-type littermates when they were fed either a regular chow diet or a high fat diet. The PIPKIIβ knockout mice did not exhibit any reduced viability or other gross physiological abnormalities.

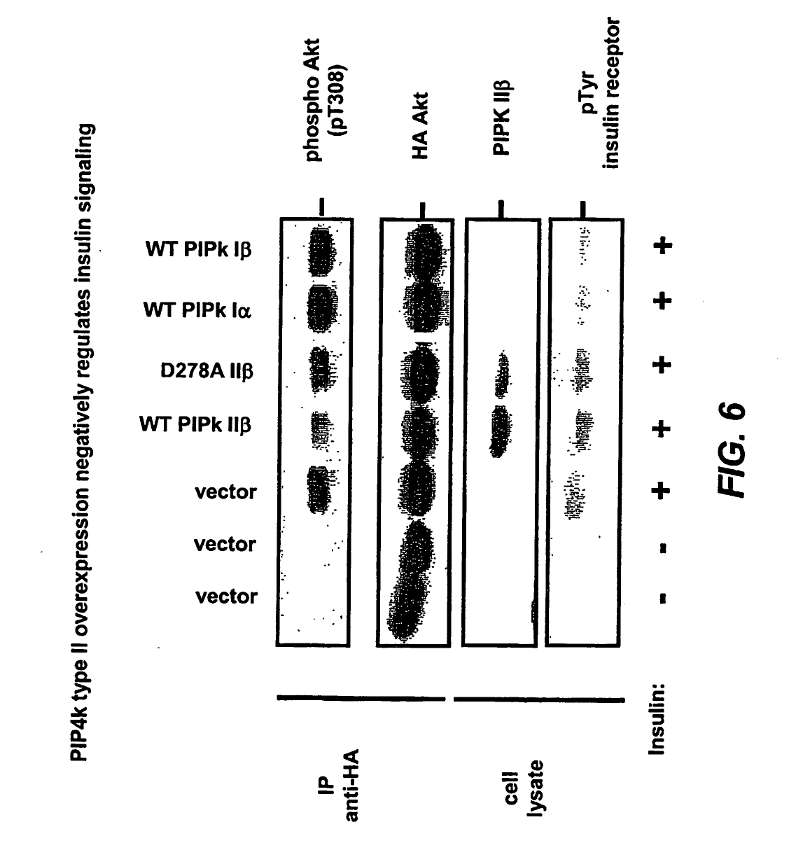

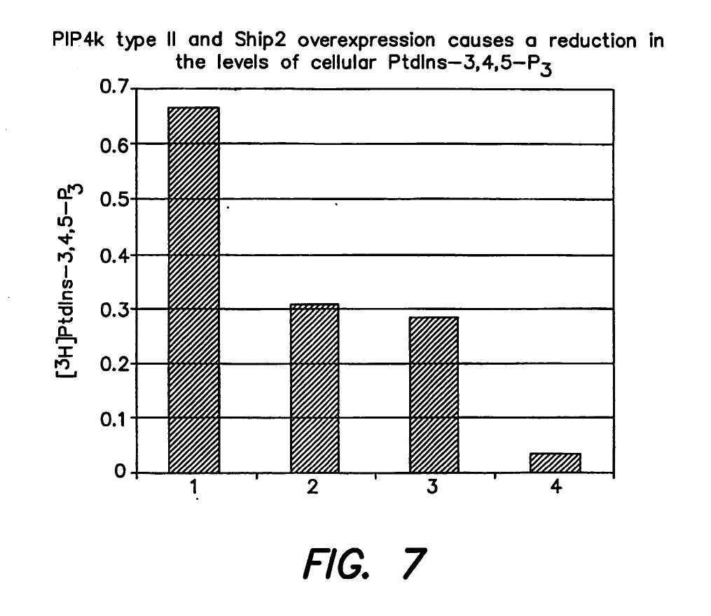

To further investigate the role of PIPKIIβ in insulin signaling, this enzyme was overexpressed in insulin-responsive CHO-IR cells. In normal CHO-IR cells, insulin stimulates the formation of a signaling complex between phosphoinositide 3-kinase (PI3K) and IRS-1/IRS-2 proteins, resulting in the production of phosphatidylinositol 3,4,5-trisphosphate (PIP₃). PIP3 recruits and activates the protein-serine/threonine kinase Akt. When PIPKIIβ is overexpressed in CHO-IR cell lines, insulin stimulation of the PI3K/IRS-1/IRS-2 signaling complex is normal, but surprisingly, activation of Akt is impaired (FIG. 6). In addition, PIP₃ levels are decreased in CHO-IR cells overexpressing PIPKIIβ, suggesting that overexpression of PIPKIIβ causes hydrolysis of PIP₃ (FIG. 7). Thus, loss of PIPKIIβ improves insulin signaling while overexpression of PIPKIIβ impairs insulin signaling.

These data suggest that drugs that specifically inhibit either the catalytic activity or the expression of PIPKIIβ (GenBank accession number NM_{—}003559) may reduce obesity and also alleviate insulin resistance in patients suffering from type II diabetes. Accordingly, the invention provides methods for identifying agents useful in treating these disorders by inhibiting the activity or expression of PIPKIIβ.

PIPKIIβ is specifically highly expressed in skeletal muscle, which is one of the major peripheral tissues that acts to clear glucose from the blood in response to insulin. PIPKIIβ is also highly expressed in brain and adipose tissue. Surprisingly, it has now been found that a decrease in PIPKIIβ activity increases insulin sensitivity and therefore is useful to treat type II diabetes and insulin insensitivity. In addition, inhibition of PIPKIIβ activity is useful to treat obesity and excess accumulation of fat. It has also been found that an increase in PIPKIIβ activity results in decreased insulin signaling. In addition, the level of expression and/or activity of PIPKIIβ nucleic acid molecules and the polypeptides they encode may be useful as markers for the onset, progression, and/or regression of PIPKIIβ-associated disorders, including, but not limited to: type II diabetes, insensitivity to insulin, excess fat accumulation, and obesity.

The identification of the effect of altered PIPKIIβ activity allows the use of pharmaceutical agents that modify the activity of PIPKIIβ in methods of treating PIPKIIβ-associated disorders including, but not limited to type II diabetes, reduced insulin sensitivity, obesity, and/or the excess accumulation of fat. In addition, determination of the levels of expression and/or activity of the PIPKIIβ nucleic acids and polypeptides they encode may be useful as diagnostic assays for PIPKIIβ-associated disorders. Assays to determine the catalytic activity of the PIPKIIβ polypeptide may be useful in methods and kits to diagnose PIPKIIβ-associated disorders. Such assays are also useful to screen candidate compounds for use in altering the activity level of PIPKIIβ polypeptide, thereby identifying pharmaceutical agents that are useful for the treatment of PIPKIW-associated disorders. Cell and tissue samples and animal models can be used for screening candidate modulators of PIPKIIβ activity. Such methods, assays and kits are also useful to detect PIPKIIβ-associated disorders in human subjects, and for staging the onset, progression, or regression of PIPKIIβ-associated disorders in subjects. In addition, the methods, assays, and kits described herein may be used to evaluate treatments for PIPKIIβ-associated disorders.

The invention described herein relates in part to the novel identification of nucleic acids and the polypeptides they encode that are aberrantly expressed in PIPKIIβ-associated disorders, including, but not limited to: type II diabetes, reduced insulin sensitivity, obesity, and/or the excess accumulation of fat.

As used herein, the term “aberrantly” means abnormally, and may include increased expression or functional activity and/or decreased expression or functional activity. The PIPKIIβ nucleic acids and the polypeptides they encode may be used as markers for PIPKIIβ-associated disorders, including, but not limited to: type II diabetes, insensitivity to insulin (also described herein as reduced sensitivity to insulin), excess fat accumulation, and obesity. In addition, the PIPKIIβ nucleic acids and the polypeptides they encode may also be used in the diagnosis and treatment assessment of PIPKIIβ-associated disorders in humans.

As used herein, “PIPKIIβ polypeptides,” means polypeptides that are encoded by PIPKIIβ nucleic acid molecules (e.g. Genbank Accession No: NM-03559). These PIPKIIβ nucleic acids and PIPKIIβ polypeptides may be aberrantly expressed in cells, tissues, or subjects with PIPKIIβ disorders. The invention also relates, in part, to the use of the nucleic acid molecules that encode the PIPKIIβ polypeptides and also relates in part to the use of the PIPKIIβ polypeptides. In all embodiments, human PIPKIIβ polypeptides and the encoding nucleic acid molecules thereof, are preferred (e.g. Genbank Accession No: NM_{—}03559). As used herein, the “encoding nucleic acid molecules thereof” means the nucleic acid molecules that code for the polypeptides. As used herein, the term “molecules” is meant to includes nucleic acid and polypeptides of the invention.

As used herein, a subject is preferably a human, non-human primate, cow, horse, pig, sheep, goat, dog, cat, or rodent. In all embodiments, human subjects are preferred. In some embodiments, the subject is suspected of having a PIPKIIβ-associated disorder and in preferred embodiments the subject is suspected of having: type II diabetes, reduced insulin sensitivity, obesity, and/or the excess accumulation of fat. In some embodiments the subject has been diagnosed with a PIPKIIβ-associated disorder, and in preferred embodiments the subject has been diagnosed with: type II diabetes, reduced insulin sensitivity, obesity, and/or the excess accumulation of fat.

Methods for identifying subjects suspected of having a PIPKIIβ-associated disorder may include but are not limited to: physical examination, subject's family medical history, subject's medical history, blood tests, visual exam, mean body mass assessment, and/or weight assessment. Diagnostic methods for PIPKIIβ-associated disorders such as type II diabetes, insulin insensitivity, obesity, and the excess accumulation of fat are well-known to those of skill in the medical arts, although not with respect to PIPKIIβ activity.

As used herein, a biological sample includes, but is not limited to: tissue, cells, or body fluid (e.g. blood or lymph node fluid). The fluid sample may include cells and/or fluid. The tissue and cells may be obtained from a subject or may be grown in culture (e.g. from a cell line). The type of biological sample may include, but is not limited to: skeletal muscle, brain, and/or adipose tissue, which is also referred to herein as “fat.” In some embodiments of the invention, the biological sample is a control sample, and the level of expression of PIPKIIβ nucleic acid molecules of the invention or PIPKIIβ polypeptides encoded by the nucleic acid molecules of the invention in such tissue is a control level.

As used herein a “control” may be a predetermined value, which can take a variety of forms. It can be a single cut-off value, such as a median or mean. It can be established based upon comparative groups, such as in groups having normal amounts of circulating insulin and groups having abnormal amounts of circulating insulin, or individuals with normal fat accumulation and individuals with excess accumulation of fat. Another example of comparative groups would be groups having a particular disease, condition or symptoms and groups without the disease, condition or symptoms. Another comparative group would be a group with a family history of a condition and a group without such a family history. The predetermined value can be arranged, for example, where a tested population is divided equally (or unequally) into groups, such as a low-risk group, a medium-risk group and a high-risk group or into quandrants or quintiles, the lowest quandrant or quintile being individuals with the lowest risk or amounts of PIPKIIβ nucleic acid expression and/or polypeptide expression and/or activity, and the highest quandrant or quintile being individuals with the highest risk or amounts of PIPKIIβ nucleic acid expression and/or polypeptide expression and/or activity.

The predetermined value, of course, will depend upon the particular population selected. For example, an apparently healthy population will have a different ‘normal’ range than will a population which is known to have a condition related to abnormal PIPKIIβ molecule expression or activity. Accordingly, the predetermined value selected may take into account the category in which an individual falls. Appropriate ranges and categories can be selected with no more than routine experimentation by those of ordinary skill in the art. By abnormally high it is meant high relative to a selected control. Typically the control will be based on apparently healthy normal individuals in an appropriate age bracket.

In some embodiments, a control sample is from a cell, tissue, or subject that does not have a PIPKIIβ-associated disorder. In other embodiments the control sample is a sample that is untreated with a candidate agent. For example, an effect of a candidate agent may be determined by determining the catalytic activity of a normal or abnormal PIPKIIβ polypeptide in advance of contacting the PIPKIIβ polypeptide with the agent, and again after contacting the PIPKIIβ, polypeptide with the agent, in which case, the initial level of catalytic activity determined may serve as a control level against with the post-contact level of catalytic activity may be compared. In such assays, the source of the PIPKIIβ polypeptide may be a biological sample known to be free of PIPKIIβ-associated disorder or may be a sample from a cell or tissue with a known PIPKIIβ-associated disorder, and in each case the before-contact determination of catalytic activity may be the control for the after-contact determination of catalytic activity.

The phrase “suspected of having a PIPKIIβ-associated disorder” as used herein means a tissue or tissue sample believed by one of ordinary skill in the art to contain aberrant levels or activity of PIPKIIβ nucleic acid molecules and/or the polypeptides they encode. Examples of methods for obtaining the sample from the biopsy include aspiration, gross apportioning of a mass, microdissection, laser-based microdissection, or other art-known cell-separation methods.

Because of the variability of the cell types in diseased-tissue biopsy material, and the variability in sensitivity of the diagnostic methods used, the sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the nucleic acid for use in the invention are well known to one of ordinary skill in the art. An example of this, although not intended to be limiting, is that in some instances a sample from the biopsy may be sufficient for assessment of RNA expression without amplification, but in other instances the lack of suitable cells in a small biopsy region may require use of RNA conversion and/or amplification methods or other methods to enhance resolution of the nucleic acid molecules. Such methods, which allow use of limited biopsy materials, are well known to those of ordinary skill in the art and include, but are not limited to: direct RNA amplification, reverse transcription of RNA to cDNA, real-tome RT-PCR, amplification of cDNA, or the generation of radio-labeled nucleic acids.

In some embodiments, the PIPKIIβ nucleic acid molecule is a nucleotide sequence set forth as SEQ ID NO: 1 and the PIPKIIβ polypeptide is encoded by the nucleotide sequence set forth as SEQ ID NO: 1, having the amino acid sequences set forth as SEQ ID NO: 2. In other embodiments, PIPKIIβ polypeptides may include polypeptides other than those encoded by nucleic acid molecules comprising a nucleotide sequence set forth as SEQ D NO:1.

TABLE 1

PIPKIIβ nucleic acid and amino acid
identification.

| | | |
|---|---|---|
| Sequence type | Accession No. | SEQ ID NO: |

| | | |
|---|---|---|
| Nucleic Acid | NM_03559 | 1 |
| Protein | NP_03550 | 2 |

The invention involves in some embodiments diagnosing or monitoring PIPKIIβ-associated disorders by determining the level of expression of a PIPKIIβ nucleic acid molecule and/or determining the presence or activity level of a PIPKIIβ polypeptide it encodes. In some important embodiments, this determination is performed by assaying a tissue sample from subject, preferably one believed to have a PIPKIIβ-associated disorder, for a level of expression of a PIPKIIβ nucleic acid molecule or for the amount of a PIPKIIβ polypeptide encoded by the nucleic acid molecule of the invention. In some embodiments, the level of catalytic activity of a PIPKIIβ polypeptide from a tissue sample from a subject can also be determined as a indicator of a PIPKIIβ-associated disorder.

The surprising discovery that PIPKIIβ activity is related to insulin sensitivity provides for novel methods of treatment of type II diabetes, insulin insensitivity, obesity, and the excess accumulation of fat, or other disorders in which aberrant PIPKIIβ activity is involved. In particular, methods for treating: type II diabetes, reduced insulin sensitivity, obesity, and/or the accumulation of fat are provided by the invention, in which PIPKIIβ activity is inhibited. Inhibition of PIPKIIβ activity decreases phosphorylation of phosphoinositides. Other physiological activities influenced by PIPKIIβ activity may also be affected by such inhibition, which can lead to desirable effects such as the aforementioned effect of increasing insulin sensitivity.

Any method for inhibiting PIPKIIβ activity will be useful in the treatment of disorders related to PIPKIIβ activity, such as type II diabetes, reduced sensitivity to insulin, excess fat accumulation, and obesity. PIPKIIβ activity can be inhibited by pharmacological inhibitors of the enzyme activity or its expression. PIPKIIβ activity also can be inhibited by other means, such as binding of anti-PIPKIIβ antibodies to inhibit its activity, expression of antisense PIPKIIβ nucleic acid molecules (including dsRNA for RNA interference with gene expression), and the like.

Treatment for a PIPKIIβ-associated disorder may include, but is not limited to: surgical intervention, dietetic therapy, and pharmaceutical therapy. In some embodiments, treatment may include administration of a pharmaceutical agent that increases insulin sensitivity of cells or tissues due to an inhibition of PIPKIIβ activity. The inhibitors of PIPKIIβ activity can be administered in conjunction with other pharmaceutical agents for treatment of type II diabetes, including other insulin sensitizers, insulin secretagogues, insulin, and the like.

In some embodiments, treatment may include administering antisense molecules to reduce expression of a PIPKIIβ nucleic acid molecule and PIPKIIβ polypeptide of the invention. In certain embodiments, treatment may include administering antibodies that specifically bind to the PIPKIIβ polypeptide. Optionally, an antibody can be linked to one or more detectable markers or immunomodulators. Detectable markers include, for example, radioactive or fluorescent markers. In other embodiments, treatments of certain conditions involving aberrant insulin signaling may include administration of a pharmaceutical agent that decreases insulin sensitivity of cells or tissues. This decrease may be due to an enhancement, or increase of PIPKIIβ activity.

The invention thus involves in one aspect, PIPKIIβ polypeptides, genes encoding those polypeptides, functional modifications and variants of the foregoing, useful fragments of the foregoing, as well as diagnostics relating thereto, and diagnostic uses thereof. In some embodiments, the PIPKIIβ polypeptide gene corresponds to SEQ ID NO: 1. Encoded polypeptides (e.g., proteins), peptides and antisera thereto are also preferred for diagnosis and correspond to SEQ ID NO: 2.

The amino acid sequences identified herein as PIPKIIβ polypeptides, and the nucleotide sequences encoding them, are sequences deposited in databases such as GenBank. The use of these identified PIPKIIβ sequences in pharmaceutical screening assays, determination of pharmaceutical agents, and diagnostic assays for PIPKIIβ-associated disorders is novel.

Homologs and alleles of the nucleic acids encoding a PIPKIIβ polypeptide of the invention can be identified by conventional techniques. Thus, an aspect of the invention is those nucleic acid sequences that code for a PIPKIIβ polypeptide and polypeptide fragments thereof including, but not limited to catalytic polypeptides and catalytic fragments thereof, and/or antigenic polypeptides and antigenic fragments thereof. As used herein, a homolog to a PIPKIIβ polypeptide is a polypeptide from a human or other animal that has a high degree of structural similarity to the identified PIPKIIβ polypeptides.

Identification of human and other organism homologs of PIPKIIβ polypeptides will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., skeletal muscle, brain, or adipose) and use the nucleic acids that encode a PIPKIIβ polypeptide identified herein to screen the library for related nucleotide sequences. The screening preferably is performed using high-stringency conditions to identify those sequences that are closely related by sequence identity. Nucleic acids so identified can be translated into polypeptides and the polypeptides can be tested for activity, for example, for catalytic activity.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of PIPKIIβ nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing. In addition to the biochemical methods identified above, bioinformatic methods (“in silico cloning”) can be used to identify PIPKIIβ homologs and alleles.

In general, homologs and alleles typically will share at least 90% nucleotide identity and/or at least 95% amino acid identity to the sequences of a PIPKIIβ nucleic acid and polypeptide, respectively, in some instances will share at least 95% nucleotide identity and/or at least 97% amino acid identity, and in other instances will share at least 97% nucleotide identity and/or at least 99% amino acid identity. The homology can be calculated using various, publicly available software tools developed by NCBI (Bethesda, Md.) that can be obtained through the Internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for PIPKIIβ polypeptide genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of PIPKIIβ polypeptide nucleic acids, Northern blot hybridizations using the foregoing conditions can be performed on samples taken from patients with a PIPKIIβ-associated disorder or subjects suspected of having a condition characterized by abnormal PIPKIIβ molecule expression of activity. Amplification protocols such as polymerase chain reaction using primers that hybridize to the sequences presented also can be used for detection of the PIPKIIβ polypeptide genes or expression thereof.

Identification of related sequences can also be achieved using polymerase chain reaction (PCR) including RT-PCR, real-time PCR, and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., skeletal muscle, brain, adipose). One also can use expression cloning utilizing the antisera to the polypeptides of the invention to identify nucleic acids that encode related antigenic proteins in humans or other species.

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating PIPKIIβ polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as catalytic activity, antigenicity, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides nucleic acid molecules that encode fragments of PIPKIIβ polypeptides.

Fragments can be used as probes in Southern and Northern blot assays to identify such nucleic acids, or can be used in amplification assays such as those employing PCR, including, but not limited to RT-PCR and real-time PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the PIPKIIβ polypeptides, useful, for example, in the preparation of antibodies, and in immunoassays. Preferred fragments are catalytically active fragments, which are recognized by substrate agents that specifically bind to a PIPKIIβ polypeptide.

The invention also permits the construction of PIPKIIβ polypeptide gene “knock-outs,” “knock-downs” (e.g., by RNA inhibition) or “knock-ins” in cells and in animals, providing materials for studying certain aspects of PIPKIIβ-associated disorders such as type II diabetes, insulin insensitivity, obesity, and the excess accumulation of fat and obesity, and for studying the effects of regulating the expression of PIPKIIβ polypeptides. For example, a knock-in mouse may be constructed and examined for clinical parallels between the model and a PIPKIIβ-associated disorder-affected mouse with upregulated expression of a PIPKIIβ polypeptide. Such a cell or animal model may also be useful for assessing candidate inhibitors of PIPKIIβ polypeptide activity, candidate agents that increase insulin sensitivity, and treatment strategies for PIPKIIβ-associated disorders. Alternative types of cell, tissue, and animal models for PIPKIIβ-associated disorders may be developed based on the invention.

The invention also provides isolated polypeptides (including whole proteins and partial proteins) encoded by the PIPKIIβ nucleic acids. Such polypeptides are useful, for example, alone or as fusion proteins to generate antibodies, and as components of an immunoassay or diagnostic assay. PIPKIIβ polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, such as PIPKIIβ fragments including catalytically active and/or antigenic peptides also can be synthesized chemically using well-established methods of peptide synthesis.

Fragments of a polypeptide preferably are those fragments that retain a distinct functional capability of the polypeptide. Functional capabilities that can be retained in a fragment of a polypeptide include catalytic activity, interaction with antibodies (e.g. antigenic fragments), interaction with other polypeptides or fragments thereof, selective binding of nucleic acids or proteins, and catalytic activity.

The skilled artisan will also realize that conservative amino acid substitutions may be made in PIPKIIβ polypeptides to provide functionally equivalent variants, or homologs of the foregoing polypeptides, i.e, the variants retain the functional capabilities of the PIPKIIβ polypeptides, such as catalytic activity or antigenicity. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the PIPKIIβ polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, one can make conservative amino acid substitutions to the amino acid sequence of the PIPKIIβ polypeptide, and still have the polypeptide retain its specific antibody-binding characteristics and/or catalytic characteristics. Alternatively, one can make catalytically inactive or less active mutants.

Conservative amino-acid substitutions in the amino acid sequence of PIPKIIβ polypeptides to produce functionally equivalent variants of PIPKIIβ polypeptides typically are made by alteration of a nucleic acid encoding a PIPKIIβ polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. USA. 82: 488-492, 1985), or by chemical synthesis of a gene encoding a PIPKIIβ polypeptide. Where amino acid substitutions are made to a small unique fragment of a PIPKIIβ polypeptide, such as an antigenic epitope recognized by autologous or allogeneic sera or cytolytic T lymphocytes, the substitutions can be made by directly synthesizing the peptide. The activity of fragments of PIPKIIβ polypeptides can be tested by cloning the gene encoding the altered PIPKIIβ polypeptide into a bacterial or mammalian expression vector, introducing the vector into an appropriate host cell, expressing the altered polypeptide, and testing for a functional capability of the PIPKIIβ polypeptides as disclosed herein. Peptides that are chemically synthesized can be tested directly for function, e.g., for catalytic activity, and/or for binding to antisera recognizing associated antigens.

The identification herein of PIPKIIβ polypeptides as involved in physiological disorders also permits the artisan to diagnose a disorder characterized by expression of PIPKIIβ polypeptides, and characterized preferably by an alteration in functional activity of the PIPKIIβ polypeptides.

The methods related to PIPKIIβ polypeptide expression involve determining expression of one or more PIPKIIβ nucleic acids, and/or encoded PIPKIIβ polypeptides and/or peptides derived therefrom and comparing the expression with that in a subject free of a PIPKIIβ-associated disorder. Such determinations can be carried out via any standard nucleic acid determination assay, including the polymerase chain reaction, or assaying with labeled hybridization probes. Such hybridization methods include, but are not limited to microarray techniques.

Determination of the catalytic activity of PIPKIIβ polypeptides for diagnostic, prognostic, and therapeutic purposes is an aspect of the invention. The catalytic activity of a PIPKIIβ polypeptide may be determined and candidate pharmaceutical agents can be tested for their ability to modify (decrease or increase) the PIPKIIβ catalytic activity. The determination that a compound modifies the PIPKIIβ catalytic activity indicates that the compound may be useful as an agent to treat PIPKIIβ-associated disorders, such as type II diabetes, insulin insensitivity, obesity, and the excess accumulation of fat. For example, the PIPKIIβ polypeptide may be contacted with a substrate of the polypeptide and the catalytic activity of the PIPKIIβ monitored and determined, then the PIPKIIβ polypeptide may be contacted with a candidate agent and the polypeptide's catalytic activity determined upon contact with the substrate. Such assays may be done in vitro and may also be useful to monitor effects of in vivo administration of catalytic activity modulators in cells or animals, including humans. In some embodiments, the above-described types of assays may be used to identify candidate agents that increase insulin sensitivity of a cell or tissue, and in other embodiments such a method may be useful to identify candidate agents that decrease insulin sensitivity in cells and tissues. A candidate agent that inhibits PIPKIIβ polypeptide catalytic activity may increase insulin sensitivity and thereby may be useful to treat PIPKIIβ-associated disorders such as type II diabetes, insulin insensitivity, obesity, and the excess accumulation of fat. An assay of the invention may also be used to identify a candidate agent that enhances PIPKIIβ polypeptide catalytic activity. Such an assay may be useful to identify candidate agents for use in treatment of PIPKIIβ-associated disorders for which an increase in PIPKIIβ polypeptide activity is observed.

The invention also involves the use of agents such as polypeptides that bind to PIPKIIβ polypeptides. Such binding agents can be used, for example, in screening assays to detect the presence or absence of PIPKIIβ polypeptides and complexes of PIPKIIβ polypeptides and their binding partners and in purification protocols to isolate PIPKIIβ and complexes of PIPKIIβ polypeptides and their binding partners. Such agents also may be used to inhibit the native activity of the PIPKIIβ polypeptides, for example, by binding to such polypeptides, and may be useful in treatment of PIPKIIβ-associated disorders.

The invention, therefore, embraces peptide binding agents which, for example, can be antibodies or fragments of antibodies having the ability to selectively bind to PIPKIIβ polypeptides. Antibodies include polyclonal and monoclonal antibodies, prepared according to conventional methodology. As used herein, PIPKIIβ antibodies, are antibodies that specifically bind to PIPKIIβ polypeptides.

Significantly, as is well known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to PIPKIIβ polypeptides, and complexes of both PIPKIIβ polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the PIPKIIβ polypeptide. This process can be repeated through several cycles of reselection of phage that bind to the PIPKIIβ polypeptide. Repeated rounds lead to enrichment of phage bearing particular sequences. DNA sequence analysis can be conducted to identify the sequences of the expressed polypeptides. The minimal linear portion of the sequence that binds to the PIPKIIβ polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the PIPKIIβ polypeptides.

Thus, the PIPKIIβ polypeptides of the invention, including fragments thereof, can be used to screen peptide libraries, including phage display libraries, to identify and select peptide binding partners of the PIPKIIβ, polypeptides of the invention. Such molecules can be used, as described, for screening assays, for purification protocols, for interfering directly with the functioning of PIPKIIβ polypeptides and for other purposes that will be apparent to those of ordinary skill in the art. For example, although not intended to be limiting, is an assay in which isolated PIPKIIβ polypeptides can be attached to a substrate (e.g., chromatographic media, such as polystyrene beads, or a filter), and then a solution suspected of containing the binding partner may be applied to the substrate. If a binding partner that can interact with PIPKIIβ polypeptides is present in the solution, then it will bind to the substrate-bound PIPKIIβ polypeptide. The binding partner then may be isolated.

As detailed herein, the foregoing antibodies and other binding molecules may be used for example, to identify tissues expressing protein or to purify protein. Antibodies also may be coupled to specific diagnostic labeling agents for imaging of cells and tissues that express PIPKIIβ polypeptides or to therapeutically useful agents according to standard coupling procedures.

The invention also includes methods to monitor the onset, progression, or regression of a PIPKIIβ-associated disorder in a subject by, for example, obtaining samples at sequential times from a subject and assaying such samples for the level of expression of PIPKIIβ nucleic acid molecules, the level of expression of PIPKIIβ polypeptide molecules, and/or the level of activity of a PIPKIIβ polypeptide. A subject may be suspected of having a PIPKIIβ-associated disorder or may be believed not to have a PIPKIIβ-associated disorder and in the latter case, the sample expression or activity level may serve as a control for comparison with subsequent samples.

Onset of a condition is the initiation of the changes associated with the condition in a subject. Such changes may be evidenced by physiological symptoms, or may be clinically asymptomatic. For example, the onset of a PIPKIIβ-associated disorder may be followed by a period during which there may be PIPKIIβ physiological changes in the subject, even though clinical symptoms may not be evident at that time. The progression of a condition follows onset and is the advancement of the physiological elements of the condition, which may or may not be marked by an increase in clinical symptoms. Onset and progression are similar in that both represent an increase in the characteristics of a disorder (e.g. expression or activity of PIPKIIβ molecules in a PIPKIIβ-associated disorder), in a cell or subject, onset represents the beginning of this disorder and progression represents the worsening of a preexisting condition.

In contrast to onset and progression, regression of a condition is a decrease in physiological characteristics of the condition, perhaps with a parallel reduction in symptoms, and may result from a treatment or may be a natural reversal in the condition. The invention also relates in part to a method of using a PIPKIIβ nucleic acid sequence in the determination of a aberrant or mutant sequence of PIPKIIβ nucleic acid in a subject. This assay may be useful for the pre-symptomatic diagnosis and prophylactic treatment of a PIPKIIβ-associated disorder.

A marker for PIPKIIβ-associated disorders may be the level or amount of specific binding of a PIPKIIβ polypeptide with an antibody, the level or amount of catalytic activity of a PIPKIIβ polypeptide, or the level of expression of a PIPKIIβ nucleic acid. Onset of a PIPKIIβ-associated disorder may be indicated by an increased amount of such a marker(s) in a subject's samples where there was less such marker(s) determined previously. For example, if a marker for a PIPKIIβ-associated disorder is determined to be at a low level in a first sample from a subject (e.g. equal or close to the level of a normal control sample), and the PIPKIIβ-associated disorder marker is determined to be present at a higher level in a second or subsequent sample from the subject, it may indicate the onset of PIPKIIβ-associated disorder.

Progression and regression of a PIPKIIβ-associated disorder may be generally indicated by the increase or decrease, respectively, of the level of a marker in a subject's samples over time. For example, if a level of a marker for a PIPKIIβ-associated disorder is determined to be present in a first sample from a subject and a higher level of a marker for a PIPKIIβ-associated disorder is determined to be present in a second or subsequent sample from the subject, it may indicate the progression of a PIPKIIβ-associated disorder. Regression of a PIPKIIβ-associated disorder may be indicated by finding that level of a marker determined to be present in a sample from a subject are is determined to be found at lower amounts in a second or subsequent sample or samples from the subject.

The progression and regression of a PIPKIIβ-associated condition may also be indicated based on characteristics of the PIPKIIβ polypeptides determined in the subject. For example, a PIPKIIβ polypeptide may be expressed at different levels at specific stages of a PIPKIIβ-associated disorder (e.g. early-stage level of PIPKIIβ polypeptides; mid-stage level of PIPKIIβ polypeptide; and late-stage level of PIPKIIβ polypeptides).

Different types of PIPKIIβ-associated disorders, including, but not limited to: type II diabetes, insulin insensitivity, obesity, and the excess accumulation of fat, may express different levels of PIPKIIβ polypeptides and the encoding nucleic acid molecules thereof, or may have different spatial or temporal expression patterns. Such variations allow PIPKIIβ-associated disorder-specific diagnosis and subsequent treatment tailored to the patient's specific condition.

The invention also relates in part to methods of treating PIPKIIβ-associated disorders such as: type II diabetes, insensitivity to insulin, obesity, and excess accumulation of fat. An “effective amount” of a drug therapy is that amount of an agent that inhibits PIPKIIβ activity that alone, or together with further doses, produces the desired response, e.g. reduction of symptoms of type II diabetes, increases sensitivity to insulin, reduction in obesity, and or reduction in excess fat accumulation.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the agent that inhibits PIPKIIβ activity (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an agent that inhibits PIPKIIβ activity for producing the desired response in a unit of weight or volume suitable for administration to a patient.

The doses of agent that inhibits PIPKIIβ activity administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver the agent that inhibits PIPKIIβ activity to a desired tissue, cell or bodily fluid. Administration includes: topical, intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of agent that inhibits PIPKIIβ activity will be known to one of ordinary skill in the art, in which the dose amount, schedule of administration, sites of administration, mode of administration (e.g., intra-organ) and the like vary from those presented herein.

Administration of agent that inhibits PIPKIIβ activity to mammals other than humans, e.g. for testing purposes or veterinary therapeutic purposes, is carried out under substantially the same conditions as described above. It will be understood by one of ordinary skill in the art that this invention is applicable to both human and animal diseases that can be treated by agent that inhibits PIPKIIβ activity. Thus this invention is intended to be used in husbandry and veterinary medicine as well as in human therapeutics.

In general, for treatments for type II diabetes, insensitivity to insulin, obesity, and excess accumulation of fat, doses of agents that inhibit PIPKIIβ activity are formulated and administered in doses between 0.2 mg to 5000 mg of the agent that inhibits PIPKIIβ. Preferably, an effective amount will be in the range from about 0.5 mg to 500 mg of the agent that inhibits PIPKIIβ according to any standard procedure in the art. Administration of agents that inhibit PIPKIIβ activity compositions to mammals other than humans, e.g. for testing purposes or veterinary therapeutic purposes, is carried out under substantially the same conditions as described above. A therapeutically effective amount typically varies from 0.01 ng/kg to about 1000 μg/kg, preferably from about 0.1 ng/kg to about 200 μg/kg and most preferably from about 0.2 ng/kg to about 20 μg/kg, in one or more dose administrations daily, for one or more days.

The pharmaceutical preparations of the invention may be administered alone or in conjunction with standard treatment(s) of PIPKIIβ-associated disorders. For example, treatment for type II diabetes with a pharmaceutical agent of the invention, may be undertaken in parallel with treatments for diabetes that is known and practiced in the art. For example, such treatments may include, but are not limited to administration of metformin, pioglitazone, and/or rosiglitazone. Other known treatments for type II diabetes include pharmaceutical agents that increases insulin release, which may include, but are not limited to sulfonylureas, nateglinide and repaglinide. In some treatment methods, sulfonylureas include, but are not limited to glibenclamide (glyburide), gliclazide and glimepiride. In some embodiments of the invention, insulin may be administered to the subject, in conjunction with the treatment methods of the invention.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. Preferred components of the composition are described above in conjunction with the description of the agent that inhibits PIPKIIβ activity of the invention.

An agent that inhibits PIPKIIβ activity composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the agent that inhibits PIPKIIβ activity, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise an agent that inhibits PIPKIIβ activity. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A long-term sustained release implant also may be used for administration of the pharmaceutical agent composition. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by unwanted PIPKIIβ activity by placing the implant near portions of a subject affected by such activity, thereby effecting localized, high doses of the compounds of the invention.

The invention also relates in part to assays used to determine the catalytic activity of a PIPKIIβ polypeptide. The PIPKIIβ polypeptide may be attached to a surface and then contacted with a substrate molecule and the level of catalytic activity of the PIPKIIβ polypeptide or fragment thereof can be monitored and quantitated using standard methods. The aforementioned assays are not intended to be limiting. Assays for catalytic activity may also be done with the components in solution, using various art-recognized detection methods, and/or other kinase assay methods known to one of ordinary skill in the art, some of which are described herein below.

The invention further provides efficient methods of identifying pharmacological agents or lead compounds for agents useful for inhibiting or monitoring kinase activity. Generally, the screening methods involve assaying for compounds which are cleaved or which inhibit or enhance phosphorylation of a substrate. Such methods are adaptable to automated, high throughput screening of compounds.

A wide variety of assays for pharmacological agents are provided, including labeled in vitro kinase phosphorylation assays, cell-based phosphorylation assays, etc. For example, in vitro kinase phosphorylation assays are used to rapidly examine the effect of candidate pharmacological agents on the phosphorylation of a substrate by, for example, PIPKIIβ or a fragment thereof. The candidate pharmacological agents can be derived from, for example, combinatorial peptide or small molecule libraries. Convenient reagents for such assays are known in the art.

In general, substrates used in the assay methods of the invention are added to an assay mixture as an isolated molecule. For use with PIPKIIβ, a preferred substrate is PI5P. The assay mixture can include detectable phosphate compounds (e.g. ³²p or ³³P), so that phosphoinositides phosphorylated by PIPKIIβ are readily detectable. Alternatively, PIPKIIβ activity on a substrate can be measured using other detectable means such as antibody capture of specific phosphorylated inositides, chromatographic means, etc.

A typical assay mixture includes a peptide having a phosphorylation site motif and a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides (e.g., kinase sites), and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid (i.e., aptamer), the agent typically is a DNA or RNA molecule, although modified nucleic acids having non-natural bonds or subunits are also contemplated.

PIPKIIβ inhibitors also can be designed using rational structure-based methods such as the methods described in PCT/US98/10876 and references described therein.

Candidate agents are obtained from a wide variety of sources including libraries of synthetic or natural compounds. For example, numerous means are available for random and directed synthesis of a wide variety of organic compounds and biomolecules, including expression of randomized oligonucleotides, random or non-random peptide libraries, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as nuclease inhibitors, antimicrobial agents, and the like may also be used.

The mixture of the foregoing assay materials is incubated under conditions whereby, but for the presence of the candidate pharmacological agent, a PIPKIIB phosphorylates a phosphorinositol substrate (for PIPKIIβ polypeptide inhibition studies). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the presence or absence of phosphorylation or binding of a substrate is detected by any convenient method available to the user. For cell free binding type assays, a separation step may be used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide is variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific binding or interaction such as salts, buffer, detergent, non-specific protein, etc. Where the solid substrate is a magnetic bead, the beads may be washed one or more times with a washing solution and isolated using a magnet.

Detection may be effected using any convenient method. The phosphorylation produces a directly or indirectly detectable product, e.g., PI5P. In the assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a substrate or inhibitor as described elsewhere herein or to the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the present invention includes automated drug screening assays for identifying compositions having the ability to inhibit phosphorylation of a substrate directly (by binding PIPKIIβ polypeptide), or indirectly (by serving as decoy substrates). The automated methods preferably are carried out in an apparatus which is capable of delivering a reagent solution to a plurality of predetermined compartments of a vessel and measuring the change in a detectable molecule in the predetermined compartments. Exemplary methods include the following steps. First, a divided vessel is provided that has one or more compartments which contain a substrate which, when exposed to PIPKIIβ, has a detectable change. The PIPKIIβcan be in a cell in the compartment, in solution, or immobilized within the compartment. Next, one or more predetermined compartments are aligned with a predetermined position (e.g., aligned with a fluid outlet of an automatic pipette) and an aliquot of a solution containing a compound or mixture of compounds being tested for its ability to inhibit PIPKIIβ kinase activity is delivered to the predetermined compartment(s) with an automatic pipette. The substrate also can be added with the compounds or following the addition of the compounds. Finally, detectable signal; emitted by the substrate is measured for a predetermined amount of time, preferably by aligning said cell-containing compartment with a detector. Preferably, the signal also measured prior to adding the compounds to the compartments, to establish e.g., background and/or baseline values. For competition assays, the compounds can be added with or after addition of a substrate or inhibitor to the PIPKIIβ polypeptide-containing compartments. One of ordinary skill in the art can readily determine the appropriate order of addition of the assay components for particular assays.

At a suitable time after addition of the reaction components, the plate is moved, if necessary, so that assay wells are positioned for measurement of signal. Because a change in the signal may begin within the first few seconds after addition of test compounds, it is desirable to align the assay well with the signal detector as quickly as possible, with times of about two seconds or less being desirable. In preferred embodiments of the invention, where the apparatus is configured for detection through the bottom of the well(s) and compounds are added from above the well(s), readings may be taken substantially continuously, since the plate does not need to be moved for addition of reagent. The well and detector device should remain aligned for a predetermined period of time suitable to measure and record the change in signal.

The apparatus of the present invention is programmable to begin the steps of an assay sequence in a predetermined first well (or rows or columns of wells) and proceed sequentially down the columns and across the rows of the plate in a predetermined route through well number n. It is preferred that the data from replicate wells treated with the same compound are collected and recorded (e.g., stored in the memory of a computer) for calculation of signal.

To accomplish rapid compound addition and rapid reading of the response, the detector can be modified by fitting an automatic pipetter and developing a software program to accomplish precise computer control over both the detector and the automatic pipetter. By integrating the combination of the fluorometer and the automatic pipetter and using a microcomputer to control the commands to the detector and automatic pipetter, the delay time between reagent addition and detector reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple signal measurements, reliability of the assays as well as the number of assays that can be performed per day are advantageously increased.

Inhibitors of PIPKIIβ-polypeptide activity identified by the methods described herein are useful to treat diseases or conditions that result from excessive or unwanted PIPKIIβ-polypeptide activity, including type II diabetes, reduced sensitivity to insulin, obesity, and/or excess accumulation of fat, etc. For treatment of such conditions, an effective inhibitory amount of a PIPKIIβ-polypeptide inhibitor is administered to a subject. The inhibitors also can be used in diagnostic applications, to detect specific PIPKIIβ-polypeptides.

The invention includes kits for assaying the presence of PIPKIIβ polypeptides. An example of a kit may include an antibody or antigen-binding fragment thereof, that binds specifically to a PIPKIIβ polypeptide. The antibody or antigen-binding fragment thereof, may be applied to a tissue or cell sample from a patient with a PIPKIIβ-associated disorder, suspected of having a PIPKIIβ-associated disorder, or believed to be free of a PIPKIIβ-associated disorder and the sample then processed to assess whether specific binding occurs between the antibody and a polypeptide or other component of the sample.

Another example of a kit of the invention, is a kit that provides components necessary to determine the level of expression of a PIPKIIβ nucleic acid molecule of the invention. Such components may include, primers useful for amplification of a PIPKIIβ nucleic acid molecule and/or other chemicals for PCR amplification.

Another example of a kit of the invention, is a kit that provides components necessary to determine the level of expression of a PIPKIIβ nucleic acid molecule of the invention using a method of hybridization.

Another example of a kit of the invention, is a kit that provides components necessary to determine the activity level of a PIPKIIβ polypeptide of the invention using a method of enzyme assay.

The foregoing kits can include instructions or other printed material on how to use the various components of the kits for diagnostic purposes.

The invention further includes nucleic acid or protein microarrays with PIPKIIβ polypeptides or nucleic acids encoding such polypeptides. In this aspect of the invention, standard techniques of microarray technology are utilized to assess expression of the PIPKIIβ polypeptides and/or identify biological constituents that bind such polypeptides. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Nucleic acid arrays, particularly arrays that bind PIPKIIβ peptides, also can be used for diagnostic applications, such as for identifying subjects that have a condition characterized by PIPKIIβ polypeptide expression.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

Targets are peptides or proteins and may be natural or synthetic. The tissue may be obtained from a subject or may be grown in culture (e.g. from a cell line).

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, Jan. 1999, the entire contents of which is incorporated by reference herein.

According to the present invention, nucleic acid microarray substrates may include but are not limited to glass, silica, aluminosilicates, borosilicates, metal oxides such as alumina and nickel oxide, various clays, nitrocellulose, or nylon. In all embodiments, a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes is a PIPKIIβ polypeptide nucleic acid molecule set forth herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

In some embodiments, one or more control nucleic acid molecules are attached to the substrate. Preferably, control nucleic acid molecules allow determination of factors such as binding characteristics, reagent quality and effectiveness, hybridization success, and analysis thresholds and success.

### EXAMPLES

### Example 1

Methods

Generation of PIPKIIβ^{-/-} Mice

Mice lacking expression of PIPKIIβ were generated from PIPKIIβ^{+/-} ES cells obtained from Lexicon Genetics, Inc. (The Woodlands, Tex.). Lexicon used a retroviral insertion gene trap strategy to disrupt genes at random in mouse embryonic stem cells of the 129Sv/Ev genetic background (Zambrowicz et al., 1998). Clone #39557 was obtained from Lexicon Genetics, which was reported to harbor a disruption of the PIPKIIβ locus. These cells were grown and expanded in our laboratory according to Lexicon's protocols and were injected into blastocysts at the Beth Israel Deaconess Transgenic Facility. Three chimeric mice were obtained and each was mated with two C57B1/6 wild type female mice to establish a colony in the C57B1/6×129Sv/Ev mixed genetic background. All experiments were performed with wild type, heterozygous, and knockout littermates derived from crosses between PIPKIIβ^{+/-} mice in the C57B1/6×129Sv/Ev genetic background.

The sequence of the PIPKIIβ mouse genomic locus was determined by assembling contigs from the Celera database. The location of the Lexicon insertion within the first intron of the PIPKIIβ locus in ES cell clone #39557 was determined by PCR. First, the location was roughly mapped by using primer pairs to amplify the following fragments from genomic DNA from wild type and knockout samples: 327-924, 887-1412, 1397-1946, 1805-2405, 2369-2883, 2913-3540, 3393-4001, 3994-4512, 4400-4998, 4984-5636, 5371-5977, 5806-6363, 6332-6833, 6808-7376, 7282-7721 where the numbers indicate the position within the first intron. This analysis indicated that the insertion was within the first 924 bases of the intron. To identify the precise location of the insertion, a forward primer from the 3′ end of the Lexicon insertion vector and a reverse primer corresponding to bases 2883-2906 of the first intron of the PIPKIIβ locus were used to amplify a ˜4 kb fragment spanning the site of insertion. This fragment was sequenced using a primer corresponding to bases 924-944 to determine the precise location of the Lexicon insertion. (position 818 where 1=A of ATG startsite for translation).

Mouse Genotyping by PCR

A set of three primers was used to amplify regions of genomic DNA present in either the wild-type samples or the knockout samples. To do so, a single antisense primer (pR) corresponding to bases 924-942 of the intron was used. Two sense primers: one is corresponding to bases 327-350 of the intron (pwtF), and the other within the 3′ end of the Lexicon insertion (pkoF) were also used. The sequence of the 3′ end of the coding portion of the second cassette of the insertion was determined by sequencing the 4-kb fragment that was obtained by PCR spanning the insertion boundary. The sequences of the primers used were:
pR: 5′-ACC ATC CCA AAG CAC CCA GGA CC-3′ (SEQ ID NO: 3), pwtF: 5′-CGT GCGT ATG CCG TCG TCG TTT CC-3′ (SEQ ID NO: 4), pkoF: 5′-AGA AGC GAG AAG CGA ACT GAT TGG-3′ (SEQ ID NO:5). The primer pair pwtF/pR was expected to amplify a fragment of 597 bp and the primer pair pkoF/pR was expected to amplify a fragment of 651 bp.
   RT-PCR

cDNA complementary to the endogenous PIPKIIβ transcript was prepared using a primer complementary to 29 bases in exon VIII of PIPKIIβ (5′-CCT CGT CCT CTG CCC GCT CCT CCA CCT CC-3′, SEQ ID NO: 6). A fragment corresponding to bases 51-902 of the endogenous coding sequence was amplified using a forward primer from exon I (5′CGC CAG CAA GAC AAG ACC AAG AAG AAG-3′, SEQ ID NO: 7) and a nested reverse primer to exon VIII (5′-CGC TCC TCC ACC TCC ATC TCC TCC-3′, SEQ ID NO: 8).

cDNA complementary to the hybrid transcript produced by splicing the first exon of PIPKIIβ to the 5′ cassette of the Lexicon retroviral insertion vector was prepared using a primer complementary to the βgeo sequence within the Lexicon vector. A fragment from the hybrid transcript was amplified using the forward primer from exon I (described above) and a nested reverse primer from the βgeo sequence in the Lexicon insertion vector (5′-GCA TCC TTC AGC CCC TTG TTG-3′, SEQ ID NO: 9).

Body Composition Analysis

Body composition analysis was performed by two methods: Dual Energy X-ray Absorption Scan (DEXAScan) was used to measure the amount of body fat in mice at 10 weeks and 26 weeks of age. Carcass analysis (saponification and subsequent assay for glycerol content) was used to measure the fat content in mice at 36 weeks of age.

DEXAScan

A PIXIMus densitometer (GE Medical Systems, Waukesha, Wis.) was used to analyze the amount of fat tissue in the PIPKIIβ^{-/-} mice. The mice were anaesthetized with ketamine/xylazine and placed on the apparatus for measurement.

Chemical Carcass Analysis

Mice were dissected to remove the contents of the stomach and intestines and the empty stomach and intestines were returned to the carcasses. The carcasses were weighed and then placed in a 60° C. oven to dry for up to 3 weeks. Carcasses were weighed on successive days to determine when the water was fully evaporated. The weight difference before and after drying was taken as the water weight of each carcass. After drying, the carcasses were saponified in a solution of 1 part 30% potassium hydroxide and 2 parts 100% ethanol in a 60° C. oven for up to 1 week. The amount of glycerol present in the resulting carcasates was determined by enzymatic conversion and calorimetric detection with the Sigma triglyceride reagent A (Sigma #337-40-A) and comparison to triglyceride standards (Sigma #339-11) (Sigma-Aldrich, St. Louis Mo.).

Insulin Tolerance Tests

Mice were placed in clean cages (without food) at 10:00 am and were injected intraperitoneally at 1:00 pm with Novolin-R (Novo Nordisk Pharmaceuticals Inc, Princeton, N.J.) at a dosage of 0.5-0.75 Units per kg of bodyweight. Blood glucose was measured by tail bleed using a One Touch Basic glucometer before the injection of insulin and at 15, 30, 45, 60, and 90 minutes following insulin injection.

Statistical Analysis

The results of the body composition analysis experiments were analyzed for statistical significance by student's t-test. The results of the insulin tolerance tests were analyzed by repeated measures ANOVA. All statistical analyses were performed with StatView software version 4.1 (StatView Software, Cary, N.C.)

Construction of PIPKIIβD278A Mutant

The catalytically impaired PIPKIIBD278A was constructed with the CLONTECH site-directed mutagenesis kit (CLONTECH Laboratories Inc., Palo Alto, Calif.). The kinase activity of bacterially expressed recombinant PIPKIIbD278A was compared to the wild-type by performing a kinase assay with a substrate of 90% phosphatidyl serine and 10% synthetic PI5P from Echelon, Inc. PIPKIIbD278A was found to have approximately 3% of the kinase activity of the wild-type PIPKIIβ.

Cell Culture and Transfection and Stimulation with Insulin

CHO-HIR cells and Cos-7 cells were grown in DMEM+10% fetal bovine serum and were transfected by DEAE/dextran transfection. Cells were serum-starved and stimulated with 10 nM insulin for 10 minutes. Cells were lysed in NP40-based lysis buffer approximately 48 hours after transfection for analysis by immunoprecipitation and Western blotting.

Western Blotting—pAkt, Anti-HA, PIPKIIb, pTyr

CHO-HIR cells were transfected with control vector or vector expressing different PIPK genes. HA-tagged Akt was transfected as a reporter gene. The cells were serum-starved and stimulated or not with 10 nM insulin for 10 minutes before lysis. Ha-Akt was immunoprecipitated from the lysates and blotted with anti-pT308 antibody in order to determine its activation state. The immunoprecipitates were also blotted with anti-Ha-Akt antibody, anti-PIPKIIβ antibody and anti-phphotryosine (pTyr) antibody.

In Vivo Labeling and HPLC

In vivo labeling was performed by growing cells in the presence of ³²P-ATP for 4 hours prior to insulin stimulation (10 min in 10 nM insulin) and lysis. Lipids were extracted with chloroform-methanol, deacylated, and subjected to high performance liquid chromatography (HPLC) for separation and detection of PIP3 levels.

Results

PIPKIIβ^{-/-} Mice

Mice lacking expression of PIPKIIβ were generated from PIPKIIβ^{+/-} ES cells obtained from Lexicon Genetics, Inc. The mice have been genotyped by PCR and disruption of PIPKIIβ gene expression was confirmed by RT-PCR analysis from multiple tissues. From our initial studies on these knockout mice, we have determined that PIPKIIβ plays a role in insulin responsiveness and body fat accumulation. PIPKIIβ^{-/-} mice are viable, fertile, and generally healthy. From crosses between PIPKIIβ^{+/-} mice, we obtained 261 offspring of which 60 (23%) were PIPKIIβ^{+/+}, 145 (55.5%) were PIPKIIβ^{+/-}, and 56 (21.5%) were PIPKIIβ^{-/-}. These numbers fall within Mendelian expectations for transmission of an autosomal gene, and suggest that disruption of PIPKIIβ gene expression does not cause lethality. PIPKIIβ^{-/-}, mice have subsequently been observed to survive to over two years of age with no obvious histopathological abnormalities.

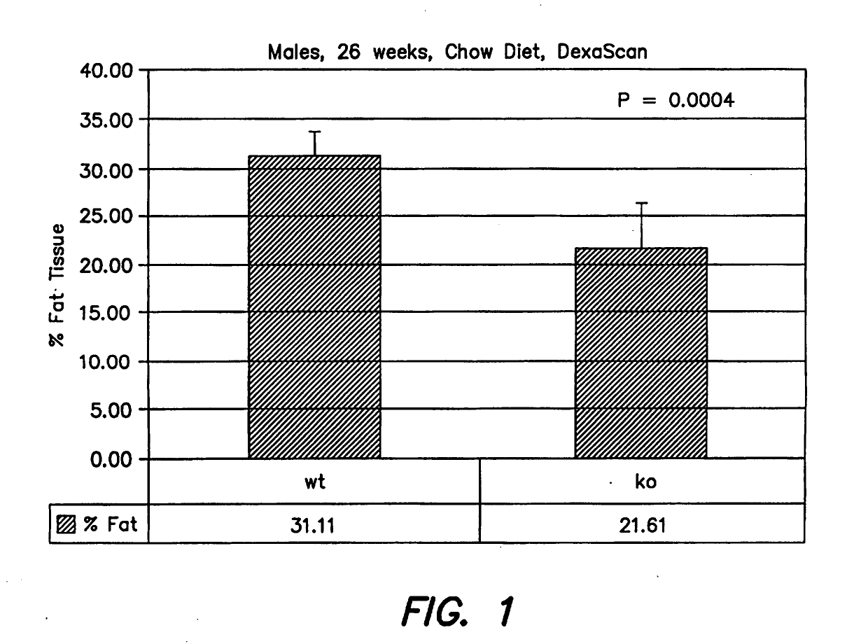

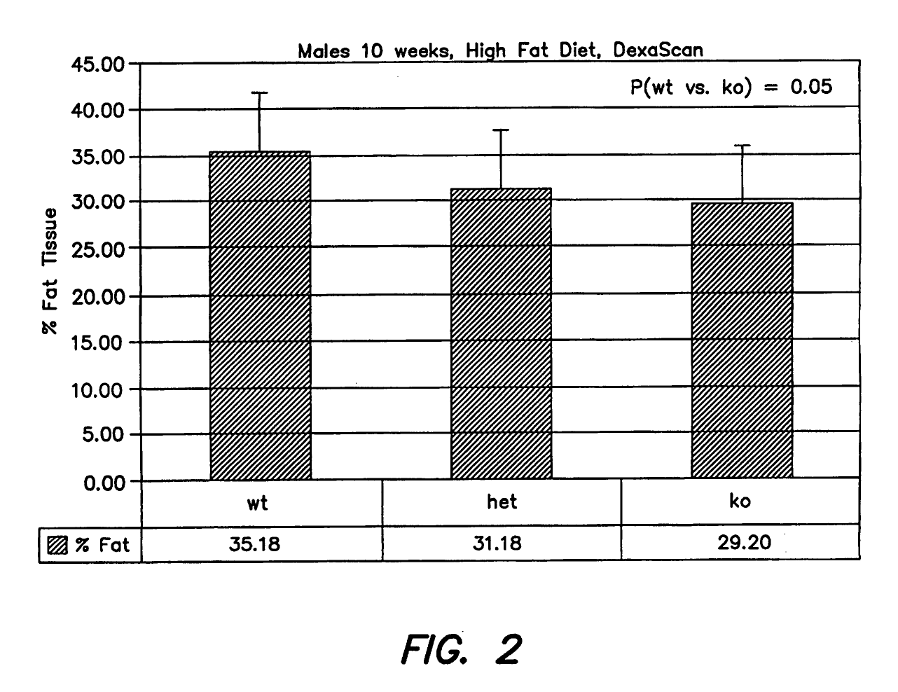

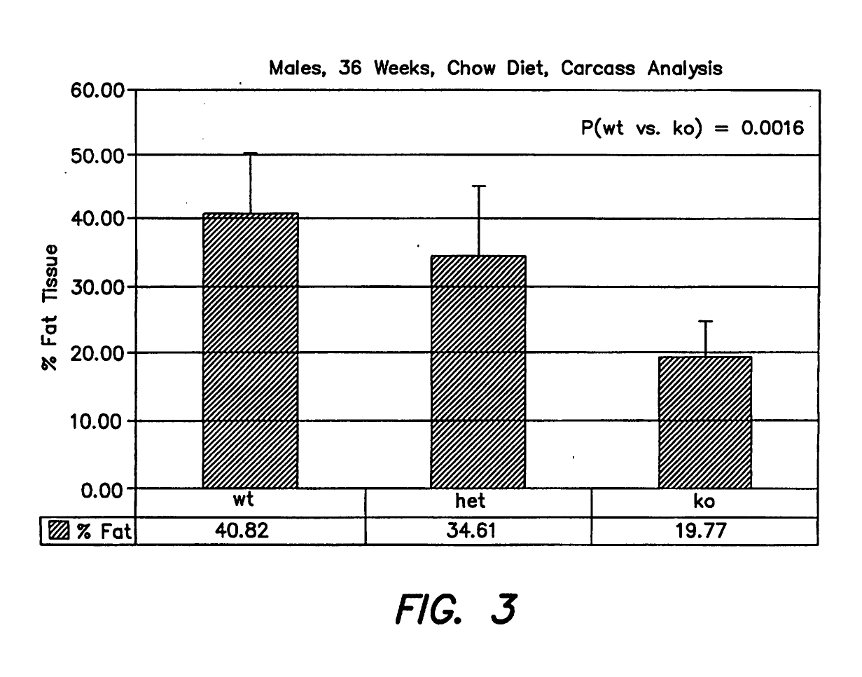

Male PIPKIIβ^{-/-} mice accumulate significantly less fat than wild type littermates when fed either a normal chow diet or a high fat diet. By weighing the mice at regular intervals, we observed that PIPKIIβ^{-/-} mice are significantly smaller than wild type littermates. To examine the reason for this difference, we used a PIXIMus densitometer to analyze the amount of fat tissue in the mice. We found that when the mice are fed a low fat (chow) diet, there is no significant difference in their body composition at 10 weeks of age. However, at 26 weeks of age PIPKIIβ^{-/-} male mice have significantly less fat than their wild type littermates (FIG. 1). When the mice are fed a high fat diet continuously from the time of weaning, the PIPKIIβ^{-/-} males exhibit a significantly lower percentage of body fat even at 10 weeks of age (FIG. 2). Body composition analysis was also performed in 36-week-old male mice by saponification and glycerol quantitation and the results were even more dramatic (FIG. 3). To determine whether the difference in fat accumulation in male mice is due to a decrease in the number of fat cells or to a decrease in the size of individual fat cells, the white fat pads are isolated and the number of cells and the amount of triglycerides in each fat pad are measured using standard protocols.

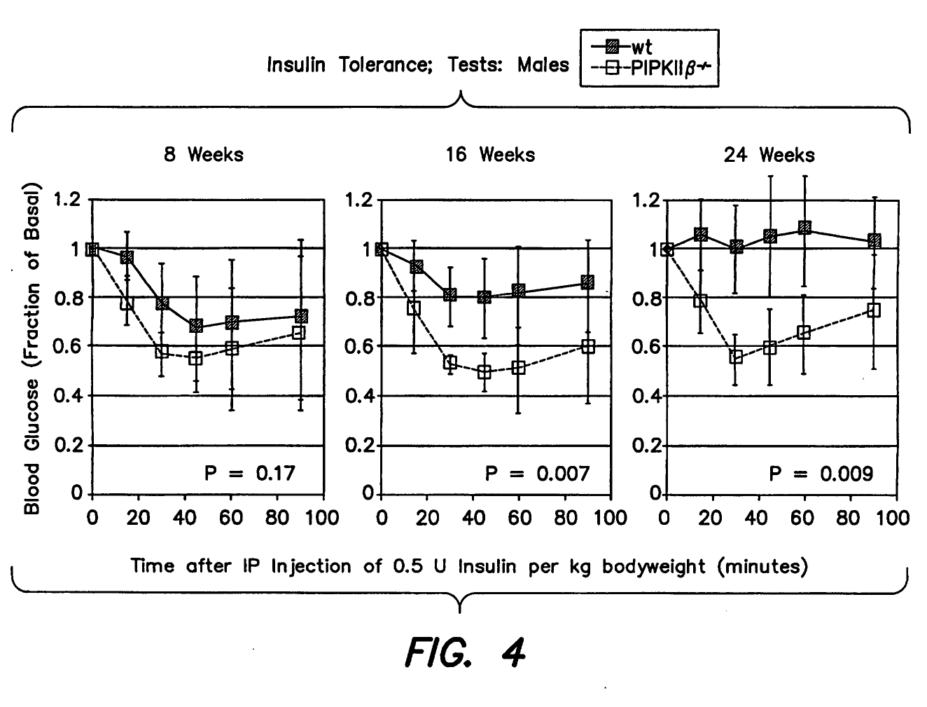

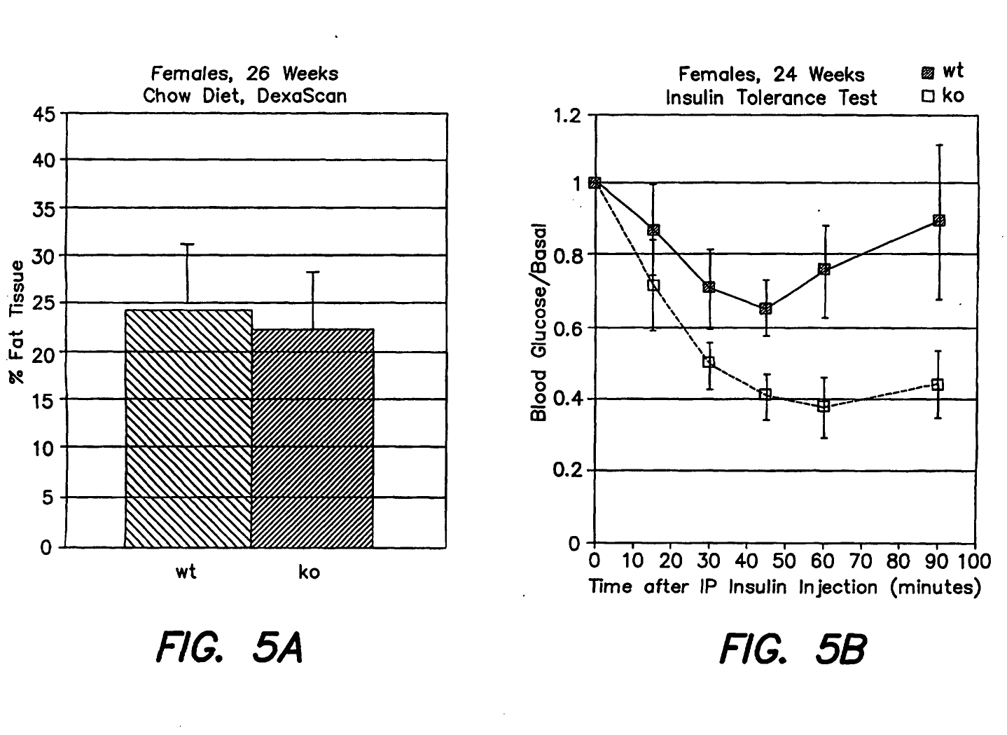

PIPKIIβ^{-/-} mice also exhibit differences in insulin responsiveness when compared to their wild type littermates. The sensitivity to insulin was assayed by performing insulin tolerance tests at 8 weeks, 16 weeks, and 24 weeks of age. We found that PIPKIIβ^{-/-} mice do not develop age-onset (i.e. type II) insulin resistance while their wild-type counterparts do (FIG. 4). The hypersensitivity to insulin observed in the knockout mice was found to be independent of the difference in fat accumulation. Female knockout mice are significantly more sensitive to insulin than their wild type littermates at 24 weeks of age while they have a similar amount of body fat (FIG. 5). To examine the effects of PIPKIIb gene expression on insulin signaling in individual tissues, euglycemic-hyperinsulinemic clamp studies are performed to measure the uptake of radiolabeled glucose into both muscle and fat tissues independently to determine whether one or both of these peripheral tissues accounts for the difference in insulin responsiveness in the whole animal. Insulin-stimulated glucose uptake in isolated muscle and fat from wild type and knockout mice are examined using standard protocols. To determine whether the effects on insulin stimulation are intrinsic to each tissue or whether they are due to the presence of a secreted factor using standard protocols. The effects of TNFα on insulin responsiveness in these mice are also determined in order to better understand the context in which PIPKIIβ effects insulin responsiveness.

Overexpression of PIPKIIβ in Cell Lines

Because deletion of PIPKIIβ increased insulin sensitivity in the mouse, we asked whether overexpression of this enzyme would affect insulin signaling in a cell line. CHO-HIR cells express the human insulin receptor and are frequently used to study intracellular signaling events downstream of the activation of the insulin receptor. We transfected CHO-HIR cells with wild type PIPKIIβ, PIPKIIβD278A (a mutant with reduced catalytic activity), or with the Type I PIP kinases, PIPKIα or PIPKIβ, or with vector alone. The transfected cells were stimulated with insulin (10 nM insulin for 10 minutes) and lysed and Ha-Akt was immunoprecipitated from the lysates and blotted with anti-pT308 antibody in order to determine its activated state. Akt was activated as judged by blotting with an antibody specific for the activated form of this enzyme (FIG. 6). However, in cells overexpressing PIPKIIβ, there was a significant reduction in the insulin-dependent activation of Akt. This effect was not as pronounced using the catalytically impaired PIPKIIβ mutant (PIPKIIβD278A). Overexpression of the type I PIP kinases PIPKIα or PIPKIβ had no effect on Akt activation. These results suggest that high levels of PIPKIIβ activity result in a block in insulin-dependent activation of AKT.

One possible mechanism for inhibition of Akt activation is a block in the formation of the PI3K-IRS-1 or PI3K-IRS-2 complex. However, we did not detect any decrease in PI3K activity in these complexes in cells overexpressing PIPKIIβ (data not shown).

Another possibility is that PIPKIIβ accelerates the degradation of the PI3K product PIP₃, which is required to activate Akt. PIP₃ is produced by PI3K in response to insulin and it is degraded by phosphatases such as SHIP2. We examined the levels of PIP₃ in Cos cells transfected with constitutively active PI3K and with PIPKIIβ and/or SHIP2. Cells were serum-starved and labeled with [³H]-inositol for 24 hours. Lipids were extracted, deacylated and PtdIns-3,4,5-P₃ levels were analyzed by HPLC. Expression of either PIPKIIβ or SHIP2 resulted in a reduction in PIP₃ and expression of both proteins resulted in almost a complete depletion of PIP3 (FIG. 7). Thus, the reduced activation of Akt in response to insulin could be explained by enhanced degradation of PIP3 in cells overexpressing PIPKIIβ. To further investigate the mechanism by which PIPKIIb affects PIP3 production, its effects on the activity and localization of the lipid phosphatase SHIP2 are determined.

In summary, deletion of PIPKIIβ in mice results in increased insulin sensitivity, while overexpression of this same enzyme in cells in culture results in a decrease in insulin activation of Akt. These results indicate that PIPKIIβ is in a pathway that negatively regulates insulin signaling. Thus, drugs that block PIPKIIβ function are effective in treating insulin resistance and diabetes. In addition, because the mice that lack PIPKIIβ have lower levels of fat, PIPKIIβ inhibitors are effective in treating obesity and reducing fat accumulation.

### REFERENCES

Cantley L C (2001) Transcription. Translocating tubby. Science 292(5524): 2019-21.
Fruman D A, Mauvais-Jarvis F, Pollard D A, Yballe C M, Brazil D, Bronson R T, Kahn C R, Cantley L C (2000) Hypoglycaemia, liver necrosis and perinatal death in mice lacking all isoforms of phosphoinositide 3-kinase p85 alpha Nat Genet 2000 26(3):379-82.
Gillooly D J, Simonsen A, Stenmark H (2001) Cellular functions of phosphoinositol 3-phosphate and FYVE domain proteins. Biochemical J. 355(Pt 2):249-58.
Hurley J H, Meyer T (2001) Subcellular targeting by membrane lipids. Curr Opin Cell Biol. 13(2):146-52.
Lemmon M A, Ferguson K M (2000) Signal-dependent membrane targeting by pleckstrin homology (PH) domains. Biochem J. 350(Pt 1):1-18.
Li J, DeFea K, Roth F A (1999) Modulation of insulin receptor substrate-1 tyrosine phosphorylation by an Akt/phosphatidylinositol 3-kinase pathway. J Biol Chem 274(14):9351-6.
Martin T F (2001) PI(4,5)P2 regulation of surface membrane traffic. Current Opinion in Cell Biology 13:493-99.
Ozes O N, Akca H, Mayo L D, Gustin J A, Maehama T, Dixon J E, Donner D B (2001) Proc Natl Acad Sci USA 98(8):4640-5.
Rameh L E, Tolias K F, Duckworth B C, and Cantley L C (1997) A new pathway for synthesis of phosphatidylinositol-4,5-bisphosphate Nature 390(6656): 192-6.
Santagata S, Boggon T J, Baird C L, Gomez C A, Zhao J, Shan W S, Myszka D G, Shapiro L (2001) G-protein signaling through tubby proteins. Science 292(5524): 2041-50.
Takenawa T, and Miki H (2001) J. Cell Science 114(Pt 10):1801-9.
Toker A (1998) The synthesis and cellular roles of phosphatidylinositol 4,5-bisphosphate. Curr Opin Cell Biol 10(2):254-61.
Wishart M J, Taylor G S, Dixon J E (2001) Phoxy lipids: revealing PX domains as phosphoinositide binding modules. Cell 105(7):817-20.
Zambrowicz B P, Friedrich G A, Buxton E C, Lilleberg S L, Person C, Sands A T (1998) Nature 392(6676):608-11.

Other aspects of the invention will be clear to the skilled artisan and need not be repeated here. Each reference cited herein is incorporated by reference in its entirety.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

We claim:

1-25. (canceled)

26. A method for identifying an agent that decreases PIPKIIβ activity, comprising:
determining a first amount of activity of a PIPKIIβ polypeptide,
contacting the PIPKIIβ polypeptide with a candidate pharmacological agent,
determining the amount of activity of the contacted PIPKIIβ polypeptide, wherein a decrease in the amount of activity of the contacted PIPKIIβ polypeptide relative to the first amount of activity of the PIPKIIβ polypeptide is an indication that the candidate pharmacological agent decreases PIPKIIβ activity.

27. The method of claim 26, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

28. The method of claim 26, wherein the PIPKIIβ polypeptide comprises an amino acid sequence set forth as SEQ ID NO: 2.

29. A method for identifying an agent that increases PIPKIIβ activity, comprising:
determining a first amount of activity of a PIPKIIβ polypeptide,
contacting the PIPKIIβ polypeptide with a candidate pharmacological agent,
determining the amount activity of the contacted PIPKIIβ polypeptide, wherein an increase in the amount of activity in the contacted PIPKIIβ polypeptide relative to the first amount of activity of the PIPKIIβ polypeptide is an indication that the candidate pharmacological agent increases PIPKIIβ activity.

30. The method of claim 29, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

31. The method of claim 29, wherein the PIPKIIβ polypeptide comprises an amino acid sequence set forth as SEQ ID NO: 2.

32. A method of diagnosing a PIPKIIβ-associated disorder in a subject comprising:
obtaining a biological sample from a subject,
determining the level of activity of a PIPKIIβ polypeptide molecule in the biological sample,
comparing the level of activity of the PIPKIIβ polypeptide molecule in the biological sample with the level of activity of a PIPKIIβ polypeptide molecule in a control tissue, wherein a higher level of activity of the PIPKIIβ polypeptide molecule in the biological sample from the subject than the activity of the PIPKIIβ polypeptide molecule in the control sample is diagnostic for a PIPKIIβ-associated disorder in the subject.

33. The method of claim 32, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

34. The method of claim 32, wherein the PIPKIIβ polypeptide comprises an amino acid sequence set forth as SEQ ID NO: 2.

35. The method of claim 32, wherein the biological sample is selected from the group consisting of: tissue and cells.

36. The method of claim 35, wherein the tissue or cells is selected from the group consisting of: skeletal muscle, brain, and adipose tissue.

37. The method of claim 32, wherein the activity is determined with a kinase assay.

38. The method of claim 32, wherein the PIPKIIβ-associated disorder is selected from the group consisting of: diabetes and obesity.

39. A method for preparing an animal model of a disorder characterized by increased activity of a PIPKIIβ molecule, comprising:
introducing into a non-human subject a PIPKIIβ molecule that increases PIPKIIβ activity.

40. The method of claim 39, wherein the PIPKIIβ molecule is a PIPKIIβ nucleic acid molecule.

41. The method of claim 40, wherein the PIPKIIβ nucleic acid molecule comprises a nucleotide sequence set forth as SEQ ID NO: 1, or having at least about 95% homology to the nucleotide sequence set forth as SEQ ID NO:1.

42. The method of claim 39, wherein the PIPKIIβ molecule is a PIPKIIβ polypeptide.

43. The method of claim 42, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

44. The method of claim 42, wherein the PIPKIIβ polypeptide comprises an amino acid sequence set forth as SEQ ID NO: 2.

45. The method of claim 39, wherein the animal model is of a disorder that is selected from the group consisting of: type II diabetes, insensitivity to insulin, excess fat accumulation, and obesity.

46. A method for preparing an animal model of a disorder characterized by decreased expression of a PIPKIIβ molecule, comprising:
introducing into a non-human subject, a mutant PIPKIIβ molecule, that decreases PIPKIIβ activity.

47. The method of claim 46, wherein the PIPKIIβ molecule is a mutant PIPKIIβ nucleic acid molecule.

48. The method of claim 46, wherein the PIPKIIβ molecule is a mutant PIPKIIβ polypeptide.

49. A method for evaluating the effect of a candidate pharmacological agent on a PIPKIIβ-associated disorder, comprising:
administering a candidate pharmaceutical agent to a subject with a PIPKIIβ-associated disorder;
determining the effect of the candidate pharmaceutical agent on the activity level of a PIPKIIβ polypeptide relative to the activity level of a PIPKIIβ polypeptide in a subject to which no candidate pharmaceutical agent is administered, wherein a relative increase or relative decrease in the activity level of the PIPKIIβ polypeptide indicates an effect of the pharmaceutical agent on the PIPKIIβ-associated disorder

50. The method of claim 49, wherein the activity level of the PIPKIIβ polypeptide is determined with a kinase assay.

51. The method of claim 49, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

52. The method of claim 49, wherein the PIPKIIβ polypeptide comprises an amino acid sequence set forth as SEQ ID NO: 2.

53. The method of claim 49, wherein the PIPKIIβ-associated disorder is selected from the group consisting of: type II diabetes, insensitivity to insulin, excess fat accumulation, and obesity.

54. A method for evaluating the effect of a candidate pharmacological agent on a PIPKIIβ-associated disorder, comprising:
administering a candidate pharmaceutical agent to a subject with a PIPKIIβ-associated disorder;
determining the effect of the candidate pharmaceutical agent on the level of expression of a PIPKIIβ molecule relative to the level of expression of a PIPKIIβ molecule in a subject to which no candidate pharmaceutical agent is administered, wherein a relative increase or relative decrease in the level of expression of a PIPKIIβ molecule indicates an effect of the pharmaceutical agent on the PIPKIIβ-associated disorder.

55. The method of claim 54, wherein the PIPKIIβ molecule is a PIPKIIβ nucleic acid molecule.

56. The method of claim 55, wherein the PIPKIIβ nucleic acid molecule comprises a nucleotide sequence set forth as SEQ ID NO: 1, or having at least about 95% homology to the nucleotide sequence set forth as SEQ ID NO:1.

57. The method of claim 54, wherein the PIPKIIβ molecule is a PIPKIIβ polypeptide.

58. The method of claim 57, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

59. The method of claim 57, wherein the PIPKIIβ polypeptide comprises an amino acid sequence set forth as SEQ ID NO: 2.

60. The method of claim 54, wherein the animal model is of a disorder that is selected from the group consisting of: type II diabetes, insensitivity to insulin, excess fat accumulation, and obesity.

61. A method of diagnosing a PIPKIIβ-associated disorder in a subject comprising:
obtaining a biological sample from a subject,
determining the level of expression of a PIPKIIβ nucleic acid molecule in the biological sample,
comparing the level of expression in the biological sample with the level of expression of the nucleic acid molecule in a control biological sample, wherein a higher level of expression of the PIPKIIβ nucleic acid molecule in the biological sample from the subject than in the control biological sample is diagnostic for a PIPKIIβ-associated disorder in the subject.

62. The method of claim 61, wherein the PIPKIIβ nucleic acid molecule comprises a nucleotide sequence set forth as SEQ ID NO: 1, or having at least about 95% homology to the nucleotide sequence set forth as SEQ ID NO:1.

63. The method of claim 61, wherein the biological sample is selected from the group consisting of: tissue and cells.

64. The method of claim 63, wherein the tissue or cells is selected from the group consisting of: skeletal muscle, brain, and adipose tissue.

65. The method of claim 61, wherein the PIPKIIβ-associated disorder is selected from the group consisting of: type II diabetes, insensitivity to insulin, excess fat accumulation, and obesity.

66. The method of claim 61, wherein the level of expression of PIPKIIβ nucleic acid molecules is determined by a method selected from the group consisting of nucleic acid hybridization and nucleic acid amplification.

67. The method of claim 66, wherein the nucleic acid hybridization is performed using a nucleic acid microarray.

68. The method of claim 66, wherein the nucleic acid amplification is selected from the group consisting of PCR, RT-PCR, and real-time PCR.

69. A method for determining progression or regression of a PIPKIIβ-associated disorder in a subject comprising:
obtaining from a subject two biological samples, wherein the samples comprise the same tissue type and are obtained at different times,
determining a level of expression of a PIPKIIβ nucleic acid molecule in the two biological samples, and
comparing the levels of expression in the two biological samples,
wherein a higher level of expression of the PIPKIIβ nucleic acid molecule in the first biological sample than in the second biological sample indicates regression of a PIPKIIβ-associated disorder,
wherein a lower level of expression of the PIPKIIβ nucleic acid molecule in the first biological sample than the second biological sample indicates progression of a PIPKIIβ-associated disorder.

70. The method of claim 69, wherein the PIPKIIβ nucleic acid molecule comprises a nucleotide sequence set forth as SEQ ID NO: 1, or having at least about 95% homology to the nucleotide sequence set forth as SEQ ID NO:1.

71. The method of claim 69, wherein the biological sample is selected from the group consisting of: tissue and cells.

72. The method of claim 71, wherein the tissue or cells is selected from the group consisting of: skeletal muscle, brain, and adipose tissue.

73. The method of claim 69, wherein the PIPKIIβ-associated disorder is selected from the group consisting of: type II diabetes, insensitivity to insulin, excess fat accumulation, and obesity.

74. The method of claim 69, wherein the level of expression of PIPKIIβ nucleic acid molecules is determined by a method selected from the group consisting of nucleic acid hybridization and nucleic acid amplification.

75. The method of claim 74, wherein the nucleic acid hybridization is performed using a nucleic acid microarray.

76. The method of claim 74, wherein the nucleic acid amplification is selected from the group consisting of PCR, RT-PCR, and real-time PCR.

77. A method of diagnosing a PIPKIIβ-associated disorder in a subject comprising:
obtaining a biological sample from a subject,
comparing the level of PIPKIIβ polypeptide in the biological sample with the level of PIPKIIβ polypeptide in a control biological sample,
wherein a level of PIPKIIβ polypeptide in the biological sample from the subject that is higher than the level of PIPKIIβ polypeptide in the control biological sample is diagnostic for a PIPKIIβ-associated disorder in the subject.

78. The method of claim 77, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

79. The method of claim 77, wherein the PIPKIIβ polypeptide comprises an amino acid sequence set forth as SEQ ID NO: 2.

80. The method of claim 77, wherein the biological sample is selected from the group consisting of: tissue and cells.

81. The method of claim 80, wherein the tissue or cells is selected from the group consisting of: skeletal muscle, brain, and adipose tissue.

82. The method of claim 77, wherein the PIPKIIβ-associated disorder is selected from the group consisting of: type II diabetes, insensitivity to insulin, excess fat accumulation, and obesity.

83. The method of claim 77, wherein the level of expression of the PIPKIIβ polypeptide is determined by a method selected from the group consisting of immunohistochemistry and immunoprecipitation.

84. A method for determining progression or regression of a PIPKIIβ-associated disorder in a subject comprising:
obtaining from a subject two biological samples, wherein the samples comprise the same tissue type and are obtained at different times,
comparing the levels of PIPKIIβ polypeptide in the two biological samples,
wherein a higher level of PIPKIIβ polypeptide in the first biological sample than in the second biological sample indicates regression of a PIPKIIβ-associated disorder,
wherein a lower level of PIPKIIβ polypeptide in the first biological sample than the second biological sample indicates progression of a PIPKIIβ-associated disorder.

85. The method of claim 84, wherein the PIPKIIβ polypeptide is encoded by a nucleic acid molecule comprising a nucleotide sequence set forth as SEQ ID NO: 1, or having at least about 95% homology to the nucleotide sequence set forth as SEQ ID NO:1.

86. The method of claim 84, wherein the PIPKIIβ polypeptide comprises an amino acid sequence set forth as SEQ ID NO: 2.

87. The method of claim 84, wherein the biological sample is selected from the group consisting of: tissue and cells.

88. The method of claim 87, wherein the tissue or cells is selected from the group consisting of: skeletal muscle, brain, and adipose tissue.

89. The method of claim 84, wherein the PIPKIIβ-associated disorder is selected from the group consisting of: type II diabetes, insensitivity to insulin, excess fat accumulation, and obesity.

90. The method of claim 84, wherein the level of expression of the PIPKIIβ polypeptide is determined by a method selected from the group consisting of immunohistochemistry and immunoprecipitation.

91. A method of diagnosing a PIPKIIβ-associated disorder in a subject comprising:
obtaining a biological sample from a subject,
determining the nucleotide sequence of a PIPKIIβ nucleic acid molecule in the biological sample,
comparing the nucleotide sequence in the subject sample with the nucleotide sequence of a control PIPKIIβ nucleic acid molecule, wherein a difference between the nucleotide sequence in the subject biological sample and the control PIPKIIβ nucleic acid molecule is diagnostic for a PIPKIIβ-associated disorder in the subject.

92. The method of claim 91, wherein the PIPKIIβ nucleic acid molecule comprises a nucleotide sequence set forth as SEQ ID NO: 1, or having at least about 95% homology to the nucleotide sequence set forth as SEQ ID NO:1.

93. The method of claim 91, wherein the biological sample is selected from the group consisting of: tissue and cells.

94. The method of claim 93, wherein the tissue or cells is selected from the group consisting of: skeletal muscle, brain, and adipose tissue.

95. The method of claim 91, wherein the PIPKIIβ-associated disorder is selected from the group consisting of: type II diabetes, insensitivity to insulin, excess fat accumulation, and obesity.
